(19) United States
(12) Patent Application Publication
(10) Pub. No.: US 2024/0337641 A1
(43) Pub. Date: Oct. 10, 2024

(54) STRAIN SENSOR FOR MONITORING PLANT ELONGATION

(71) Applicant: THE BOARD OF TRUSTEES OF THE UNIVERSITY OF ILLINOIS, URBANA, IL (US)

(72) Inventors: Ying Diao, Urbana, IL (US); Siqing Wang, Urbana, IL (US)

(21) Appl. No.: 18/629,876

(22) Filed: Apr. 8, 2024

Related U.S. Application Data
(60) Provisional application No. 63/495,105, filed on Apr. 9, 2023.

(51) Int. Cl. G01N 33/00; C08K 3/28; C08K 3/30; C08K 3/38; C08K 5/3445; G01L 1/22

(52) U.S. Cl. CPC G01N 33/0098; C08K 3/28; C08K 3/30; C08K 3/38; C08K 5/3445; G01L 1/2293

(57) ABSTRACT

This present disclosure is directed strain sensors for monitoring e.g., plant growth.

### CROSS REFERENCE TO RELATED APPLICATIONS

This application claims the benefit of the filing date of U.S. Provisional Application No. 63/495,105, filed on Apr. 9, 2023, entitled STRAIN SENSOR FOR MONITORING PLANT ELONGATION. The entirety of the foregoing is hereby incorporated by reference.

### STATEMENT OF FEDERALLY SPONSORED RESEARCH AND DEVELOPMENT

This invention was made with government support under Grant No. 80NSSC21K0070 awarded by NASA. The government has certain rights in the invention.

### FIELD OF THE INVENTION

The field of the invention relates generally to systems and method for monitoring plant growth.

### BACKGROUND

Plants make up above 80% of biomass on planet earth and underpin the vitality of our ecosystem. However, rising temperature and extreme weather are putting plants under an unprecedented level of stress from more frequent and prolonged droughts, pest and pathogen infections, and wildfires. Besides the urgent need to protect ecological plants, there is a daunting task of increasing agricultural plant productivity to feed a growing human population—by 2050, the global food demand is projected to increase by as much as 60%. Moreover, future human colonization of space critically depends on the ability to cultivate vegetables under extraterrestrial conditions, as highlighted in the movie Martian. Important to addressing these challenges is the ability to precisely, continuously and autonomously monitor plant growth—a critical indicator to plant health and productivity. However, current methods are limited in meeting this need. For instance, the direct measurement of plant mass or tissue elongation are often intermittent and require extensive human efforts. On the other hand, plant tracking technologies using camera imaging usually require bulky and costly instrumentation and infrastructure. Furthermore, the two-dimensional nature of camera imaging and fixed camera positions lead to low measurement precision given the three-dimensional and dynamic nature of plant growth. Therefore, new technologies are urgently needed to achieve real-time, remote, autonomous, and precise measurement of plant growth.

Wearable electronics are attractive for addressing the above challenges due to their flexibility, surface conformability, and compatibility with wireless data transmission systems. Wearable strain sensors, wherein mechanical deformation is transduced to electrical signals, have been extensively applied to human motion detection, human health monitoring, structural health monitoring, soft robotics, human-machine interfaces and electronic skin. However, this technology has been rarely applied to plant growth monitoring. This is because tracking plant growth imposes challenging requirements on device performance, including high stretchability, large strain sensing range, minimal interference with plant growth and photosynthesis, and stable performance under complex environmental conditions. The reported wearable plant strain sensors based on gold, carbon nanotubes, or carbon-based composites suffer from low transparency to light and exhibit limited strain sensing ranges (<120%). Besides, due to their heavy weight and large modulus, these sensors have largely been applied to mature and mechanically resilient plants and tissues, such as cucumber fruits, stems of bamboo or tomato, maize leaves, etc. So far, strain sensing of more delicate plant tissues remains challenging. Comparing to the aforementioned hard materials, conjugated-polymer-based organic electronics have been designed to make soft, lightweight, stretchable, and transparent devices including strain sensors, stretchable electrodes, and supercapacitors. However, these stretchable organic electronic devices have not been applied to monitor plant growth because of the challenges in environmental stability, response reliability, and reproducibility.

Traditionally, the technologies used to monitor plant growth include manual measurement or camera inspection, as well as measuring growth related parameters such as gas exchanges and chlorophyll a fluorescence. However, these methods are either too crude or too complex to detect plant growth remotely and continuously. Other strain sensors developed for plant growth monitoring have limited strain sensing range (less than 150%) and therefore can only monitor plant growth rate in a short time within the plant life, ranging from 20 minutes to several days. There is need for a sensor that can measure plant growth rate with minimal human intervention while not impeding the plant growth.

To address this need the inventors have fabricated strain sensors using materials such as a highly stretchable substrate, a linear responsive strain sensing film and a stiff adhesive layer. These strain sensors have a minimal effect of plant growth, and can sense a strain of as high as 750%. Leaf growth was successfully tracked on grass for 26 hours and on Mizuna for 9 days.

The strain sensors for plant monitoring disclosed herein may be included as part of a smart agriculture system. The strain sensor will act as a terminal on the plant side and provide information of plant growth directly to the control end with minimal or without human intervention.

### DESCRIPTION

### Definitions

For the purposes of promoting an understanding of the principles of the invention, reference will now be made to certain embodiments and specific language will be used to describe the same. It will nevertheless be understood that no limitation of the scope of the invention is thereby intended, and alterations and modifications in the illustrated invention, and further applications of the principles of the invention as illustrated therein are herein contemplated as would normally occur to one skilled in the art to which the invention relates.

Unless defined otherwise, all technical and scientific terms used herein have the same meaning as commonly understood by one of ordinary skill in the art to which this invention pertains.

For the purpose of interpreting this specification, the following definitions will apply and whenever appropriate, terms used in the singular will also include the plural and vice versa. In the event that any definition set forth below conflicts with the usage of that word in any other document, including any document incorporated herein by reference, the definition set forth below shall always control for purposes of interpreting this specification and its associated claims unless a contrary meaning is clearly intended (for example in the document where the term is originally used).

The use of “or” means “and/or” unless stated otherwise.

The use of “a” or “an” herein means “one or more” unless stated otherwise or where the use of “one or more” is clearly inappropriate.

The use of “comprise,” “comprises,” “comprising,” “include,” “includes,” and “including” are interchangeable and not intended to be limiting. Furthermore, where the description of one or more embodiments uses the term “comprising,” those skilled in the art would understand that, in some specific instances, the embodiment or embodiments can be alternatively described using the language “consisting essentially of” and/or “consisting of.”

As used herein, the term “about” refers to a ±10% variation from the nominal value. It is to be understood that such a variation is always included in any given value provided herein, whether or not it is specifically referred to.

Any ranges given either in absolute terms or in approximate terms are intended to encompass both, and any definitions used herein are intended to be clarifying and not limiting. Notwithstanding that the numerical ranges and parameters setting forth the broad scope of the invention are approximations, the numerical values set forth in the specific examples are reported as precisely as possible. Moreover, all ranges disclosed herein are to be understood to encompass any and all subranges (including all fractional and whole values) subsumed therein.

The term “plant” or “plants” refers to the eukaryotes that form the kingdom Plantae which are predominantly photosynthetic, meaning that they obtain their energy from sunlight, using chloroplasts derived from endosymbiosis with cyanobacteria to produce sugars from carbon dioxide and water, using the green pigment chlorophyll. Exceptions are parasitic plants that have lost the genes for chlorophyll and photosynthesis and obtain their energy from other plants or fungi.

As used herein “stretchability” refers generally to the formula below:

Stretchability=(Largest working length−original length)/original length
Stretchability is measured by the largest strain of operation, which is defined as the change of length over the original length. For example, 1% means that the SSF layer can be stretched to 101% of its original length, and 1000% means that the SSF layer can be stretched to 1100% of its original length.

As used herein, the term “transparency” refers generally to transmittance of light and is measured by UV-Vis spectroscopy within the wavelength range of 300 nm to 1000 nm.

As used herein, the term “vias” refers to holes in the encapsulation layer that allows external electrical connection to the SSF layer.

### Terminology

ADC Analog-to-Digital Converter
ARMS Autonomous Resistance Measurement System
COS crack onset strains
CV coefficient of variation
DR degradation rate
GIWAXS Grazing-Incidence Wide-Angle X-ray Scattering.
LL Landau-Levich regime
MGP meniscus-guided printing technique
OTS treated glass: “oxidized titanium” or “oxidized tin” treated glass
PEDOT: PSS Poly(3,4-ethylenedioxythiophene) polystyrene sulfonate
Prostat PRS-801 a wide range resistance meter
rDoC the relative degree of crystallinity
SEBS styrene-ethylene-butylene-styrene
SPEARS² Stretchable, Polymer-based, Electronic, Autonomous, and Remote Strain Sensor
SSF a strain sensing film
PSS polystyrene sulfonate
SSR a strain sensing range
SNAP Subnetwork Access Protocol
SWCNT single walled carbon nanotubes
WST water-soluble tape

While plants represent ˜82% of the total biomass on earth and are critical to climate change mitigation, terrestrial and extraterrestrial agriculture, the direct and precise measurement of plant growth with minimal human intervention has been challenging. Wearable strain sensors can help address this issue due to their flexibility, surface conformability, and compatibility with wireless data transmission systems. However, the few reported wearable strain sensors for monitoring plant growth have limited strain sensing range (<120%), are not transparent to light, and there is a lack of study on stability and reproducibility. These limitations severely hinder their application in precisely tracking large plant elongation. The inventors have developed transparent conjugated polymer-based strain sensors that achieves a strain sensing range of e.g., ˜700%. Without wishing to be limited by any particular theory, by leveraging the confinement effect attained through thin film printing, film crystallinity and crack development are suppressed, which increases the strain sensing range by x fold. For instance, through device engineering, a strain sensor which is ultra-lightweight (˜45 mg), shows high transparency (transmittance=98.7%) and environmental stability (degradation rate=0.0008 h-1), and exhibits excellent response linearity (R2=0.996) and good reproducibility (coefficient of variation=14.4%), may be fabricated, which are crucial factors for precisely and robustly monitoring plant growth. Combining the strain sensor with a custom-built wireless autonomous resistance measurement system, remote and autonomous tracking of plant growth may be achieved. This will enable those in the agriculture fields to unveils the circadian rhythm of leaf elongation which is challenging to obtain using current methods.

One aspect of the invention pertains to a strain sensor for plants, said sensor comprising
a substrate layer,
a strain sensing film (SSF) layer, wherein said SSF layer comprises a conductive polymer,
a surfactant, and an ionic additive,
an encapsulation layer,
at least one electrode material,
one or more pastes (e.g., a silver (Ag) paste).
one or more wires (e.g., a silver (Ag) flexible wire).
one or more adhesive layers
wherein said SSF layer has a stretchability of about 1 to about 1000% and a transparency of 0% to about 99%.

Another aspect of the invention pertains to a strain sensor, said sensor comprising:
a substrate layer,
a strain sensing film (SSF) layer, wherein said SSF layer comprises a conductive polymer, optionally, a surfactant, and optionally an ionic additive,
an encapsulation layer,
at least one electrode material,
one or more pastes (e.g., a silver (Ag) paste),
one or more wires (e.g., a silver (Ag) flexible wire),
one or more adhesive layers,
wherein said SSF layer has a stretchability of about 1-1000% and a transparency of 70% to 99%.

A further aspect of the invention pertains to a strain sensor, said sensor comprising:
a substrate layer,
a strain sensing film (SSF) layer, wherein said SSF layer comprises a conductive polymer, a surfactant, and an ionic additive,
an encapsulation layer,
at least one electrode material,
one or more pastes (e.g., a silver (Ag) paste),
one or more wires (e.g., a silver (Ag) flexible wire),
one or more adhesive layers,
wherein said SSF layer has a stretchability of about 200-1000% and a transparency of 0% to 99%.

A yet aspect of the invention pertains to a strain sensor, said sensor comprising:
a substrate layer,
a strain sensing film (SSF) layer, wherein said SSF layer comprises a conductive polymer, a surfactant, and an ionic additive,
an encapsulation layer,
at least one electrode material,
one or more pastes (e.g., a silver (Ag) paste),
one or more wires (e.g., a silver (Ag) flexible wire),
one or more adhesive layers,
wherein said SSF layer has a stretchability of about 200-1000% and a transparency of about 70% to about 99%.

In some embodiments, the strain sensor disclosed herein includes a SSF layer with a stretchability of about 1-1000% and a transparency of 70% to 99%.

In some embodiments, the strain sensor disclosed herein includes SSF layer with a stretchability of about 200-1000% and a transparency of 0% to 99%.

In some embodiments, the strain sensor disclosed herein includes SSF layer with a stretchability of about 200-1000% and a transparency of about 70% to about 99%.

A strain sensor, said sensor comprising:
a styrene-ethylene-butylene-styrene (SEBS) substrate layer
a strain sensing film (SSF) layer, wherein said SSF comprises a conductive polymer, a
surfactant, and an ionic additive,
an encapsulation layer comprising SEBS,
at least one electrode material comprising a composite of poly(3,4-ethylenedioxythiophene) polystyrene sulfonate (PEDOT: PSS) and single walled carbon
nanotube (SWCNT);
one or more pastes,
one or more wires,
one or more adhesive layers,
wherein said conductive polymer comprises a poly(3,4-ethylenedioxythiophene)
polystyrene sulfonate (PEDOT: PSS),
wherein said surfactant comprises Zonyl,
wherein said ionic additive comprises Li: TFSI, EMIM:TFSI, EMIM:DCI, EMIM:DCA,
and/or EMIM:TCB, and
wherein said SSF layer has a stretchability of about 1-1000% and a transparency of 0% to 99%.

Another aspect of the invention pertains to method of monitoring elongation of a plant, said method comprising adding to a strain sensor according to any of the preceding embodiments to the plant and monitoring plant growth.

Another aspect of the invention pertains to a method of fabricating a strain sensor for monitoring a plant elongation, said method comprising:
(a) spin coating a layer of a SEBS substrate on a slide,
(b) blade coating a layer of SSF onto said SEBS layer, wherein said SSF layer is optionally subjected to an annealing process or a solution treatment process,
(c) optionally blade coating a second SEBS layer on said SSF layer,
(d) attaching an electrode material to said second SEBS layer,
(e) optionally applying said electrode on a paste and wherein said paste is attached to said second SEBS layer,
(f) peeling an assembled film off from said slide with a water-soluble tape (WST), wherein said assembled film comprises said SEBS substrate layer, said SSF layer, and said electrode,
(g) cutting said assembled film into strips,
(h) optionally pasting said strips on an adhesive layer, wherein said adhesive layers is an adhesive layer with a water-soluble tape (WST), and optionally removing said adhesive layers, and
(i) connecting said electrode with a conductive wire for resistance measurement.
   wherein said electrode comprises PEDOT: PSS, Li: TFSI, single-walled carbon nanotubes (SWCNT), or any combination thereof.

A further aspect of the invention pertains to a strain sensor, said sensor comprising:
a substrate layer,
a strain sensing film (SSF) layer, wherein said SSF layer comprises a conductive polymer, optionally, a surfactant, and optionally an ionic additive,
an encapsulation layer,
at least one electrode material,
one or more pastes (e.g., a silver (Ag) paste),
one or more wires (e.g., a silver (Ag) flexible wire),
one or more adhesive layers,
   wherein said substrate layer comprises one or more materials chosen from Table 1 (see Substrate examples).
   and strain sensing film (SSF) layer comprises one or more materials chosen from Table 1 (see Strain sensing film (SSF) layer examples).
wherein said SSF layer has a stretchability of about 1-1000% and a transparency of 70% to 99%.

TABLE 1
Summary of generally examples of materials that may be used
in fabricating strain sensors encompassed by the invention.

| Type of | | strain sensing film (SSF) | Min/max | Min/max |
|---|---|---|---|---|
| Sensor | Substrate (examples) | layer | transparency | stretchability |
| Very | Polydimethylsiloxane | Conductive polymer: | 70%/99% | 1%/1000% |
| transparent | (PDMS), | poly(3,4- | | |
| strain sensor | Polyethylene (PE), | ethylenedioxythiophene) | | |
| | Polyethylene | polystyrene sulfonate | | |
| | Terephthalate (PET), | (PEDOT:PSS), poly(3,4- | | |
| | Polypropylene (PP), | ethylenedioxythiophene) | | |
| | Polystyrene (PS), | (PEDOT) derivatives and | | |
| | Natural Rubber, | copolymers, poly(3,4- | | |
| | Styrene-ethylene- | propylenedioxythiophene) | | |
| | butylene-styrene | (PProDOT) derivatives and | | |
| | (SEBS), Ecoflex, | copolymers | | |
| | Polyether Block | Optionally, an ionic additive | | |
| | Amide (PEBA), | and a surfactant. | | |
| | Thermoplastic | | | |
| | Polyurethane (TPU), | | | |
| | Thermoplastic | | | |
| | Vulcanizate (TPV) | | | |
| Very | Natural Rubber, | Conductive polymer: | 0%/99% | 200%/1000% |
| stretchable | Polyethylene (PE), | poly(3,4- | | |
| strain sensor | Styrene-ethylene- | ethylenedioxythiophene) | | |
| | butylene-styrene | polystyrene sulfonate | | |
| | (SEBS), Ecoflex, | (PEDOT:PSS), poly(3,4- | | |
| | Polyether Block | ethylenedioxythiophene) | | |
| | Amide (PEBA), | (PEDOT) derivatives and | | |
| | Thermoplastic | copolymers, poly(3,4- | | |
| | Polyurethane (TPU), | propylenedioxythiophene) | | |
| | Thermoplastic | (PProDOT) derivatives and | | |
| | Vulcanizate (TPV) | copolymers, poly(3,4- | | |
| | | alkylenedioxythiophene)s | | |
| | | (e.g., poly(3,4- | | |
| | | dialkylthiophene)s, poly(3,4- | | |
| | | cycloalkylthiophene)s, | | |
| | | poly(3,4- | | |
| | | dialkoxythiophene)s, | | |
| | | poly(3,4- | | |
| | | alkylenedioxythiophene)s) | | |
| | | derivatives and copolymers, | | |
| | | polyaniline (PANI), | | |
| | | polythiophene (PTh), | | |
| | | Polypyrrole (PPy) | | |
| | | Ionic additives: inorganic | | |
| | | salts (e.g., NaClO₄, LiClO₄), | | |
| | | organic salts (e.g., | | |
| | | Bis(trifluoromethane)sulfoni | | |
| | | mide lithium salt, 4-(3- | | |
| | | Butyl-1-imidazolio)-1- | | |
| | | butanesulfonic acid triflate, | | |
| | | 1-Butyl-3- | | |
| | | methylimidazolium octyl | | |
| | | sulfate, Zinc | | |
| | | di[bis(trifluoromethyl | | |
| | | sulfonyl)imide], 4-(3-Butyl- | | |
| | | 1-imidazolio)-1- | | |
| | | butanesulfonate, 1-Ethyl-3- | | |
| | | methylimidazolium- | | |
| | | bis(trifluoromethylsulfonyl)i | | |
| | | mide, Methyl- | | |
| | | trioctylammonium | | |
| | | bis(trifluoromethylsulfonyl | | |
| | | imide, Trihexyltetradecyl | | |
| | | phosphonium bis(244- | | |
| | | trimethylpentyl)phosphinate, | | |
| | | 1-Butyl-3- | | |
| | | methylpyridiniumbis(trifluor | | |
| | | methylsulfonyl)imide, | | |
| | | Dioctyl | | |
| | | sulfosuccinatesodium salt, | | |
| | | Sodium | | |
| | | dodecylbenzenesulfonate, | | |
| | | Dodecylbenzenesulfonic | | |
| | | acid, 1-Ethyl-3- | | |
| | | methylimidazolium 4,5- | | |
| | | dicyanoimidazolate, 1-Ethyl- | | |
| | | 3-methylimidazolium | | |
| | | dicyanamide, 1-Ethyl-3- | | |
| | | methylimidazolium | | |
| | | tetracyanoborate) | | |
| | | Surfactants: ionic surfactants | | |
| | | (e.g., Sodium lauryl sulfate | | |
| | | (SLS), Sodium laureth | | |
| | | sulfate (SLES), Ammonium | | |
| | | lauryl sulfate (ALS), | | |
| | | Ammonium laureth sulfate | | |
| | | (ALES), Sodium stearate, | | |
| | | Sodium Dodecyl Sulfate | | |
| | | (SDS), Potassium cocoate), | | |
| | | non-ionic surfactants (e.g., | | |
| | | Zonyl, Triton X, Tween, | | |
| | | polysorbates, sorbitans, | | |
| | | PEG). | | |
| strain sensor | Natural Rubber, | Conductive polymer: | 70%/99% | 200%/1000% |
| that is | Polyethylene (PE), | poly(3,4- | | |
| stretchable | Styrene-ethylene- | ethylenedioxythiophene) | | |
| and | butylene-styrene | polystyrene sulfonate | | |
| transparent | (SEBS), Ecoflex, | (PEDOT:PSS), poly(3,4- | | |
| | Polyether Block | ethylenedioxythiophene) | | |
| | Amide (PEBA), | (PEDOT) derivatives and | | |
| | Thermoplastic | copolymers, poly(3,4- | | |
| | Polyurethane (TPU), | propylenedioxythiophene) | | |
| | Thermoplastic | (PProDOT) derivatives and | | |
| | Vulcanizate (TPV) | copolymers | | |
| | | Ionic additives: inorganic | | |
| | | salts (e.g., NaClO₄, LiClO₄), | | |
| | | organic salts (e.g., | | |
| | | Bis(trifluoromethane)sulfoni | | |
| | | mide lithium salt, 4-(3- | | |
| | | Butyl-1-imidazolio)-1- | | |
| | | butanesulfonic acid triflate, | | |
| | | 1-Butyl-3- | | |
| | | methylimidazolium octyl | | |
| | | sulfate, Zinc | | |
| | | di[bis(trifluoromethyl | | |
| | | sulfonyl)imide], 4-(3-Butyl- | | |
| | | 1-imidazolio)-1- | | |
| | | butanesulfonate, 1-Ethyl-3- | | |
| | | methylimidazolium- | | |
| | | bis(trifluoromethylsulfonyl)i | | |
| | | mide, Methyl- | | |
| | | trioctylammonium | | |
| | | bis(trifluoromethylsulfonyl | | |
| | | imide, Trihexyltetradecyl | | |
| | | phosphonium bis(244- | | |
| | | trimethylpentyl)phosphinate, | | |
| | | 1-Butyl-3- | | |
| | | methylpyridiniumbis(trifluor | | |
| | | methylsulfonyl)imide, | | |
| | | Dioctyl | | |
| | | sulfosuccinatesodium salt, | | |
| | | Sodium | | |
| | | dodecylbenzenesulfonate, | | |
| | | Dodecylbenzenesulfonic | | |
| | | acid, 1-Ethyl-3- | | |
| | | methylimidazolium 4,5- | | |
| | | dicyanoimidazolate, 1-Ethyl- | | |
| | | 3-methylimidazolium | | |
| | | dicyanamide, 1-Ethyl-3- | | |
| | | methylimidazolium | | |
| | | tetracyanoborate) | | |
| | | Surfactants: ionic surfactants | | |
| | | (e.g., Sodium lauryl sulfate | | |
| | | (SLS), Sodium laureth | | |
| | | sulfate (SLES), Ammonium | | |
| | | lauryl sulfate (ALS), | | |
| | | Ammonium laureth sulfate | | |
| | | (ALES), Sodium stearate, | | |
| | | Sodium Dodecyl Sulfate | | |
| | | (SDS), Potassium cocoate), | | |
| | | non-ionic surfactants (e.g., | | |
| | | Zonyl, Triton X, Tween, | | |
| | | polysorbates, sorbitans, | | |
| | | PEG). | | |

### BRIEF DESCRIPTION OF THE FIGURES

The following drawings form part of the present specification and are included to further demonstrate certain aspects of the present invention. The invention may be better understood by reference to one or more of these drawings in combination with the detailed description of specific embodiments presented herein.

FIG. 1A. Layer-by-layer structure of the SPEARS² on a target plant surface.

FIG. 1B. Materials system for fabricating strain sensing films.

FIG. 2A. Strain sensing curves (i.e., ln(R/R0)-strain curve) of five different spin-coated SSFs. The crack onset strain of each film was marked with X on the horizontal axis.

FIG. 2B. The crack morphology of each film when stretched to a certain strain (percentage in yellow) observed under a dark field optical microscope. Scale bar: 20 μm. The stretching and resistance measurement direction is along the short axis of the page.

Five SSFs fabricated by spin-coating on SEBS substrates from inks with different solution compositions (A, B, C, D, E, SI, Table 3) and their performances were compared. As shown in FIG. 2a.

FIGS. 3A-F. Comparison of the strain sensing performance of SSFs blade coated at 5 different speeds. The crack onset strain (COS) of each film was marked with “x” on the horizontal axis. Figures B-F displays the crack morphology of exemplary films in the crack propagation stage observed under AFM (height profile. Scale bar: 5 μm). The printing, stretching, and resistance measurement is along the horizontal direction of the AFM images.

FIG. 4A. Relationship between strain sensing performance (Gₑₓₚ and SSR), film thickness, and printing speed.

FIG. 4B. Printing speed dependence of crack onset strain (COS) and crystallinity where crystallinity is characterized by two metrics: relative degree of crystallinity (rDoc) of PEDOT π-π stacking (010) peak, and π-π/amorphous peak area ratio at chi near 90°.

FIG. 4C. Proposed confinement mechanism of how printing speed determines the final strain sensing performance by controlling film thickness, crack morphology, and film crystallinity.

FIG. 5A. SPEARS² fabrication process

FIG. 5B. Step-stretching test of three types of SPEARS². Sensors were stretched 0.5 mm (an increase of 12.5% strain for a 4 mm-long sensor) at the end of every 12 hours while the resistance was continuously measured with ARMS.

FIG. 5C. Comparison of the strain sensing performance of silver SPEARS² and black SPEARS² at a low stretching rate (0.01 mm/min)

FIG. 5D. Histogram showing distribution of Gₑₓₚ of different SPEARS². The Gₑₓₚ distribution of SPEARS² is right-shifted and broader (1.53±0.22) compared to that of Control 1 (1.17±0.15) and Control 2 (1.13±0.13).

FIGS. 6A-D. Growth tracking performance of a 4.5-mm SPEARS² on grass tested with wired ARMS.

FIG. 6A. Schematic showing grass growth and SPEARS² stretching mechanism.

FIG. 6B. Camera images of mounted SPEARS² during grass growth.

FIG. 6C. Time-dependent sensor response compared to grass growth determined from camera images (Inset: strain sensing curve of the attached SPEARS²)

FIG. 6D. The measured growth rate calculated from SPEARS² response and true growth rate measured from camera images.

FIG. 7. GIXD patterns of SSFs blade coated at different speeds (φ=90°)

FIG. 8A. Wired ARMS circuit for single sensor measurement based on Arduino.

FIG. 8B. Real picture for a wired ARMS circuit for single sensor measurement.

FIG. 8C. Wired ARMS circuit for double sensor measurement.

FIG. 8D. Real picture for a wired ARMS circuit for double sensor measurement. Resistance values: R1=10 kΩ, R2=30 kΩ, R3=100 kΩ, R4=300 kΩ, R5=1 MΩ, R6=3 MΩ, R7=10 MΩ, R8=30 MΩ, R9=100 MΩ, R10=300 MΩ, R11=1 GΩ.

FIG. 9A. Wireless ARMS circuit based on a SNAP wireless module.

FIG. 9B. Real picture of wireless ARMS circuit. Resistance values: R₁=3 MΩ, R₂=10 MΩ, R₃=30 MΩ, R₄=100 MΩ, R₅=300 MΩ, R₆=1 GΩ (c) Resistance-ADC calibration curve of each pin in the wireless ARMS.

FIG. 9C. The measured ADC (Analog to Digital Converter) values are translated into resistance values through calibration curves.

FIG. 10. The transmission data of the SPEARS² in the range of 300 nm to 1000 nm

FIGS. 11A-C. Growth tracking performance of an 8.4-mm SPEARS² on grass tested with the wireless ARMS. FIG. 11(a) shows pictures of the SPEARS² stretched by grass growth at different time points. FIG. 11(b) shows change of sensor response and grass growth with time. FIG. 11(c) shows strain sensing performance of the SPEARS² on grass.

FIG. 12. Arrangement of components of an exemplary strain sensor.

FIGS. 13A-F. Stability performance under periodic light/dark condition of the plant strain sensor with silver paste as the electrode cured at (FIG. 13a) Room Temperature (FIG. 13b) 40° C. (FIG. 13c) 120° C. The red boxes indicate the time period when the sensor is placed under light. The numbers indicate the degradation rate of the sensor under light (unit: 0.001 h-1). Microscope images of the electrode/film interface of the plant strain sensor with silver paste as the electrode cured at (FIG. 13d) Room Temperature (FIG. 13e) 40° C. (FIG. 13f) 120° C.

FIGS. 14A-E. Top: (FIG. 14a) Fabrication process of the wrapped-Al sensor to minimize the damage to the electrode. White: Glass substrate. Yellow: Double-sided tape. Gray: Aluminum foil. Orange: 3M adhesive. Green and Blue: SEBS and strain sensing film. Black: Electrode materials. Bottom: Stability performance under periodic light/dark condition of the plant strain sensor with different electrode materials: (FIG. 14b) Gold (FIG. 14c) Gold+Silver paste (FIG. 14d) Graphite ink (FIG. 14e) Gold+Graphite ink. The red boxes indicate the time period when the sensor is placed under light. The numbers indicate the degradation rate of the sensor under light (unit: 0.001 h-1)

FIGS. 15A-C. FIG. 15(a). Photos taken at different time when tracking Mizuna leaf growth with a wrapped-Al strain sensor. Width of the Al wires: 3.5 mm. FIG. 15(b). Changes of the sensor response and the strain of the sensor over a tracking period of around 75 hours. FIG. 15(c). Strain sensing performance of the wrapped-Al strain sensor on the Mizuna leaf.

FIGS. 16A-C. FIG. 16(a). Photos taken at different time when tracking tomato fruit growth with a wrapped-Al strain sensor. Width of the Al wires: 3.5 mm. FIG. 16(b). Changes of the sensor response of the experimental group (sensor on the tomato) and the control group (sensor on the ground) over a tracking period of around 8 days. FIG. 16(c). Subtraction of the experimental group and the control group over the tracking period.

The followings is a non-limiting list of embodiments:
1. A strain sensor, said sensor comprising:
   a substrate layer,
   a strain sensing film (SSF) layer, wherein said SSF layer comprises a conductive polymer, a surfactant, and an ionic additive,
   an encapsulation layer,
   at least one electrode material,
   one or more pastes (e.g., a silver (Ag) paste),
   one or more wires (e.g., a silver (Ag) flexible wire),
   one or more adhesive layers,
   wherein said SSF layer has a stretchability of about 1 to about 1000% and a transparency of 0% to about 99%.
2. A strain sensor, said sensor comprising:
   a substrate layer,
   a strain sensing film (SSF) layer, wherein said SSF layer comprises a conductive polymer, optionally, a surfactant, and optionally an ionic additive,
   an encapsulation layer,
   at least one electrode material,
   one or more pastes (e.g., a silver (Ag) paste),
   one or more wires (e.g., a silver (Ag) flexible wire),
   one or more adhesive layers,
   wherein said SSF layer has a stretchability of about 1-1000% and a transparency of 70% to 99%.
3. A strain sensor, said sensor comprising:
   a substrate layer,
   a strain sensing film (SSF) layer, wherein said SSF layer comprises a conductive polymer, a surfactant, and an ionic additive,
   an encapsulation layer,
   at least one electrode material,
   one or more pastes (e.g., a silver (Ag) paste),
   one or more wires (e.g., a silver (Ag) flexible wire),
   one or more adhesive layers,
   wherein said SSF layer has a stretchability of about 200-1000% and a transparency of 0% to 99%.
4. A strain sensor, said sensor comprising:
   a substrate layer,
   a strain sensing film (SSF) layer, wherein said SSF layer comprises a conductive polymer, a surfactant, and an ionic additive,
   an encapsulation layer,
   at least one electrode material,
   one or more pastes (e.g., a silver (Ag) paste),
   one or more wires (e.g., a silver (Ag) flexible wire),
   one or more adhesive layers,
   wherein said SSF layer has a stretchability of about 200-1000% and a transparency of about 70% to about 99%.
5. A strain sensor, said sensor comprising:
   a styrene-ethylene-butylene-styrene (SEBS) substrate layer
   a strain sensing film (SSF) layer, wherein said SSF comprises a conductive polymer, a surfactant, and an ionic additive,
   an encapsulation layer comprising SEBS,
   at least one electrode material comprising a composite of poly(3,4-ethylenedioxythiophene) polystyrene sulfonate (PEDOT: PSS) and single walled carbon nanotube (SWCNT);
   one or more pastes (e.g., a silver (Ag) paste),
   one or more wires (e.g., a silver (Ag) flexible wire or silver conductive paper, which may be made by spin coating silver paste on a paper),
   one or more adhesive layers (e.g., a removable medical adhesive),
   wherein said conductive polymer comprises a poly(3,4-ethylenedioxythiophene) polystyrene sulfonate (PEDOT: PSS),
   wherein said surfactant comprises Zonyl,
   wherein said ionic additive comprises Li: TFSI, EMIM:TFSI, EMIM:DCI, EMIM:DCA, and EMIM:TCB, and
   wherein said SSF layer has a stretchability of about 1-1000% or 200-1000% and a transparency of 0% to 99% or about 70-99%.
6. The sensor of claim 1, wherein said substrate layer comprises one or more components chosen from Polydimethylsiloxane (PDMS), Polyethylene (PE), Polyethylene Terephthalate (PET), Polypropylene (PP), Polystyrene (PS), Natural Rubber, Styrene-ethylene-butylene-styrene (SEBS), Ecoflex, Polyether Block Amide (PEBA), Thermoplastic Polyurethane (TPU), and Thermoplastic Vulcanizate (TPV).
7. The sensor of claim 1, wherein said conductive polymer is chosen from poly(3,4-ethylenedioxythiophene) polystyrene sulfonate (PEDOT: PSS), poly(3,4-ethylenedioxythiophene) (PEDOT) derivatives and copolymers, poly(3,4-propylenedioxythiophene) (PProDOT) derivatives and copolymers, poly(3,4-alkylenedioxythiophene)s (e.g., poly(3,4-dialkylthiophene)s, poly(3,4-cycloalkylthiophene)s, poly(3,4-dialkoxythiophene)s, poly(3,4-alkylenedioxythiophene) s) derivatives and copolymers, polyaniline (PANI), polythiophene (PTh), Polypyrrole (PPy)
8. The sensor of claim 1, wherein said ionic additive is chosen from inorganic salts (e.g., NaClO₄, LiClO₄), organic salts (e.g., Bis(trifluoromethane) sulfonimide lithium salt, 4-(3-Butyl-1-imidazolio)-1-butanesulfonic acid triflate, 1-Butyl-3-methylimidazolium octyl sulfate, Zinc di[bis(trifluoromethyl sulfonyl)imide], 4-(3-Butyl-1-imidazolio)-1-butanesulfonate, 1-Ethyl-3-methylimidazolium-bis(trifluoromethylsulfonyl)imide, Methyl-trioctylammonium bis(trifluoromethylsulfonyl imide, Trihexyltetradecyl phosphonium bis(2-(4-trimethylpentyl)phosphinate, 1-Butyl-3-methylpyridiniumbis(trifluormethylsulfonyl)imide, Dioctyl sulfosuccinatesodium salt, Sodium dodecylbenzenesulfonate, Dodecylbenzenesulfonic acid, 1-Ethyl-3-methylimidazolium 4,5-dicyanoimidazolate, 1-Ethyl-3-methylimidazolium dicyanamide, and 1-Ethyl-3-methylimidazolium tetracyanoborate).
9. The sensor of claim 1, wherein said surfactant is chosen from ionic surfactants (e.g., Sodium lauryl sulfate (SLS), Sodium laureth sulfate (SLES), Ammonium lauryl sulfate (ALS), Ammonium laureth sulfate (ALES), Sodium stearate, Sodium Dodecyl Sulfate (SDS), Potassium cocoate), and non-ionic surfactants (e.g., Zonyl, Triton X, Tween, polysorbates, sorbitans, PEG).
10. The sensor of claim 1, wherein said encapsulation layer comprises one or more components chosen from Polydimethylsiloxane (PDMS), Polyethylene (PE), Polyethylene Terephthalate (PET), Polypropylene (PP), Polystyrene (PS), Natural Rubber, Styrene-ethylene-butylene-styrene (SEBS), Ecoflex, Polyether Block Amide (PEBA), Thermoplastic Polyurethane (TPU), and Thermoplastic Vulcanizate (TPV).
11. The sensor of claim 1, wherein said electrode material is chosen from poly(3,4-ethylenedioxythiophene) polystyrene sulfonate (PEDOT: PSS), poly(3,4-ethylenedioxythiophene) (PEDOT) derivatives and copolymers, poly(3,4-propylenedioxythiophene) (PProDOT) derivatives and copolymers, carbon based materials (e.g., carbon nanotubes, carbon black, graphite, graphene), metals (e.g., silver, copper, gold), and the mixture thereof.
12. The sensor of claim 1, wherein said paste is a conductive paste (e.g. a paste comprising a conductive material such as silver, carbon, copper, or gold).
13. The sensor of claim 1, wherein said wire is a conductive wire (e.g. a wire comprising a metal such as silver, carbon, copper, gold, aluminum, or platinum; metal conductive paper, which may be made by spin coating metal paste on a paper, for instance, silver conductive paper).
14. The sensor of claim 1, wherein said adhesive layer (e.g., double sided flexible tapes, medical adhesives, or other tapes; adhesive layer may be removable).
15. A method for monitoring a plant elongation, said method comprising: attaching a strain sensor of any of the preceding embodiments onto a plant tissue, and monitoring plant growth by measuring a resistance of said strain sensor. In some embodiments, the sensor is attached using a stretchable adhesive. Plant growth is monitored by methods known in the art. Target plants include, without limitation, cotton, okra, soybean, cacao, kenaf and kola nut, coffee, tobacco, potato, tomato, sweet potato, rapeseed, wheat, corn, rice, barley, sorghum, grass, sugarcane, bamboo, buckwheat, snap bean, dry bean, canola, peas, peanuts, safflower, sunflower, alfalfa hay, clover, vetch, and trefoil, blackberry, blueberry, currant, elderberry, gooseberry, huckleberry, loganberry, raspberry, strawberry, grape, garlic, leek, onion, shallot, citrus hybrid, grapefruit, kumquat, lime, orange, pummelo, cucumber, melon, gourd, pumpkin, squash, eggplant, sweet pepper, hot pepper, tomatillo, herb, spice, mint, arugula, celery, chervil, endive, fennel, lettuce, parsley, radicchio, rhubarb, spinach, swiss chard, broccoli, brussels sprout, cabbage, cauliflower, collard, kale, kohlrabi, mustard green, asparagus, pear, quince, beet, sugarbeet, red beet, carrot, celeriac, chicory, horseradish, parsnip, radish rutabaga, salsify, and turnips, maple, pine, rye, millet, apricot, cherry, nectarine, peach, plum, prune, almond, beech nut, Brazil nut, butternut, cashew, chestnut, filbert, hickory nut, macadamia nut, pecan, pistachio, walnut, artichoke, cassava, and ginger plants. The plant may be a monocot or a dicot.
16. The method of claim 15, wherein said monitor occurs remotely.
17. A method of fabricating a strain sensor for monitoring a plant elongation, said method comprising:
   a) adding substrate to a treated solid substrate surface (such as glass slide, metals (e.g., silver, aluminum, iron, copper), silicon, silicon dioxide, silicone, etc.). For example, the surface may be treated to decrease the adhesion between the surface and the substrate (e.g. adding a self-assembled monolayer on a glass slide);
   b) adding at least one SSF layer on top of the substrate;
   c) placing one or more placeholders on the SSF. Typically, these placeholders are used for electrical connection. In general, the placeholders may be placed on the SSF in pairs. In some instances, a gap of a certain length (based on application) that determines the original length of the strain sensor between each pair of placeholders;
   d) fabricating an encapsulation layer is fabricated on top of the SSF and the placeholder of step (c);
   e) removing the placeholders (e.g., mechanically peeling off), taking off the encapsulation layer on top and leaving vias in the encapsulation layer;
   f) tailoring encapsulated SSF into desired shape and transferred to a substrate (which may be soft). Examples include Polydimethylsiloxane (PDMS), Polyethylene (PE), Polyethylene Terephthalate (PET), Polypropylene (PP), Polystyrene (PS), Natural Rubber, Styrene-ethylene-butylene-styrene (SEBS), Ecoflex, Polyether Block Amide (PEBA), Thermoplastic Polyurethane (TPU), Thermoplastic Vulcanizate (TPV);
   g) electrode materials are filled in the vias.
   h) conductive wires are connected to the electrode material with conductive paste.
18. A method of fabricating a strain sensor for monitoring a plant elongation, said method comprising:
   (a) spin coating a layer of a SEBS substrate on a slide, wherein said slide is an OTS treated glass slide,
   (b) blade coating a layer of SSF onto said SEBS layer, wherein said SSF layer is optionally subjected to an annealing process or a solution treatment process,
   (c) optionally blade coating a second SEBS layer on said SSF layer,
   (d) attaching an electrode material to said second SEBS layer,
   (e) optionally wherein said electrode is applied on an Ag paste and wherein said Ag paste is attached to said second SEBS layer,
   (f) peeling an assembled film off from said slide with a water-soluble tape (WST), wherein said assembled film comprises said SEBS substrate layer, said SSF layer, and said electrode,
   (g) cutting said assembled film into strips,
   (h) optionally pasting said strips on an adhesive layer, wherein said adhesive layers is an adhesive layer (such as 3M adhesive layer) with a water-soluble tape (WST), and optionally removing said adhesive layers, and
   (i) connecting said electrode with a conductive wire for resistance measurement. wherein said electrode is applied on the bare part of said second SEBS layer, wherein said SSF layer is partially covered in the middle section of said SSF layer, wherein said second SEBS layer functions as an encapsulation layer to the said SSF layer to shield against adverse effects,
      wherein said adverse effects comprise an influence from the intrinsic conductivity of the plant leaf, humidity in the air, a direct contact between said strain sensor and a plant, or any combination thereof,
      wherein said electrode comprises materials made of PEDOT: PSS, Li: TFSI, single-walled carbon nanotubes (SWCNT), or any combination thereof.

### EXAMPLES

The following examples are provided solely to illustrate the present invention and are not intended to limit the scope of the invention, described herein.

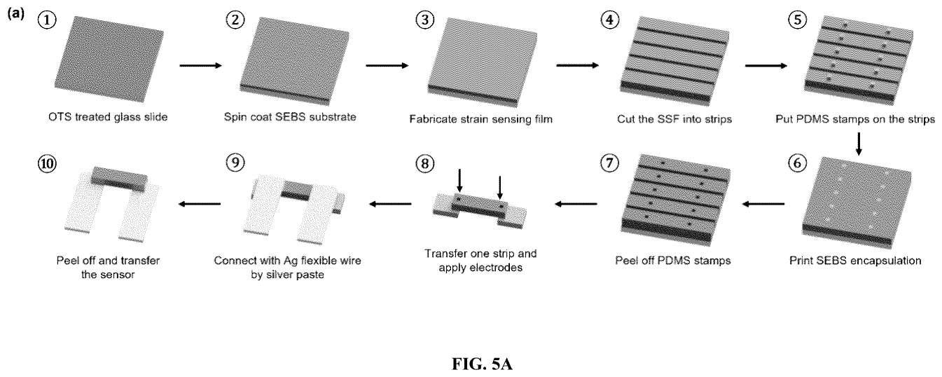

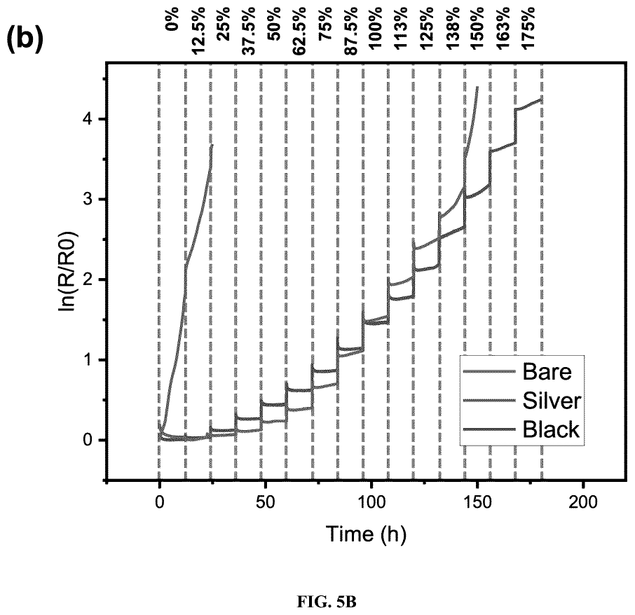

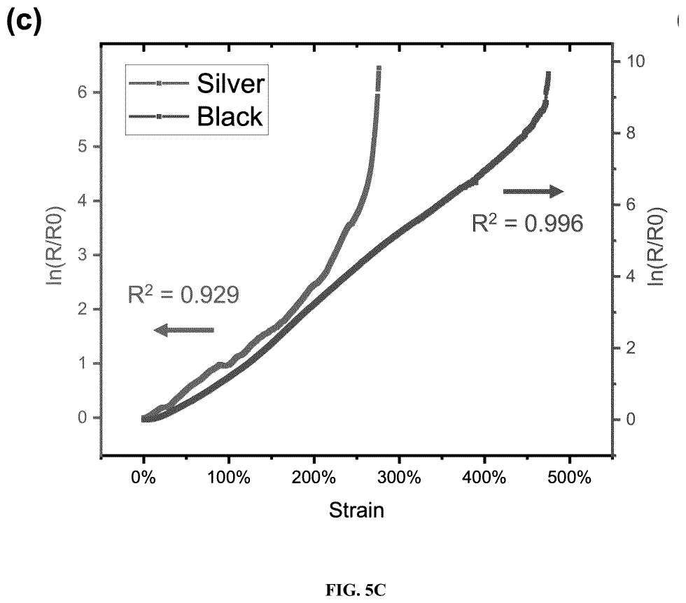

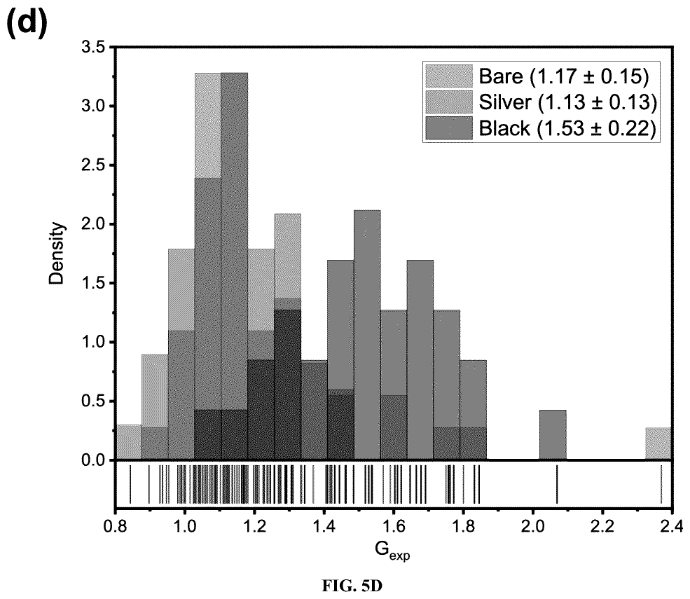

Example 1. A general procedure for fabrication can be found in the section entitled “SPEARS² Device Engineering and Testing” (especially FIG. 5a), and “SSF fabrication” and “SPEARS² Fabrication” section in methods.

A substrate will be fabricated on a surface treated (such as a glass slide) first. Then, the SSF layer will be fabricated on top of the substrate. Next, placeholders are placed on certain locations of the SSF which will be used for electrical connection later. Next, an encapsulation layer is fabricated on top. Next, the placeholders are mechanically peeled off, taking off the encapsulation layer on top and leaving vias in the encapsulation layer. Next, the encapsulated SSF is tailored into desired shape and transferred to a soft substrate. Next, electrode materials are filled in the vias. Finally, conductive wires are connected to the electrode with conductive paste.

### Example 2. General Method for Monitoring Plant Growth

When applied to plant growth monitoring, the electrode part of the sensors disclosed herein may be pasted on the plant with adhesives, leaving the middle part free-standing. Without wishing to be limited by any particular theory, when the plant grows, the sensor will stretch and induce an electrical change, which can be measured. From the measurement of electrical signal, the growth of the plant can be tracked. In some embodiments, the strain sensor is attached on the plant tissue by the adhesive layer, and the resistance of the strain sensor is measured with some resistance measurement equipment when the plant tissue is growing. The resistance change of the strain sensor can then reflect the growth of the plant tissue.

Example 3. It has been found for the first time a conjugated polymer-based wearable strain sensor termed SPEARS² (Stretchable-Polymer-Electronics-based Autonomous Remote Strain Sensor), which enables continuous, precise, plant growth monitoring of delicate grass leaves over large elongational span.

Specifically, a conductive polymer (PEDOT: PSS) based material system was utilized for fabricating a strain sensing film (SSF) with high stretchability (>700%) and transparency (optical transmittance ˜98.7%). To achieve high environmental stability, reliability, and reproducibility, an innovative device engineering and fabrication process was designed for SPEARS². A customized Autonomous Resistance Measurement System (ARMS) was then developed for autonomous and remote electrical signal monitoring. Integrating all of the above technologies resulted in a successful method to monitor plant growth continuously, remotely, and autonomously, and the ability to unveil nuanced changes in growth rate over extended periods.

### Example 4. SPEARS² Device Concept, Material and System Design

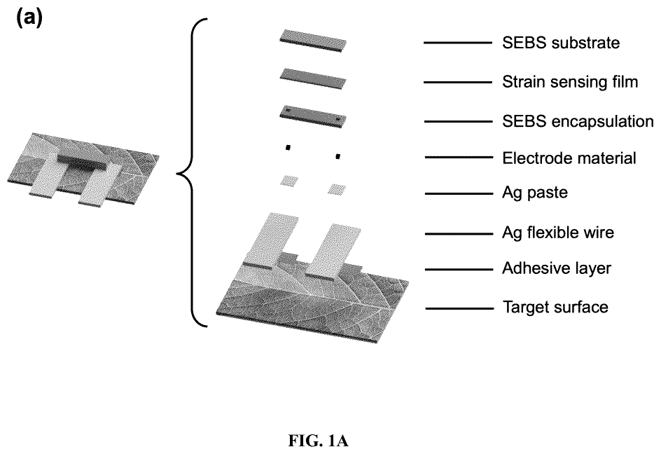

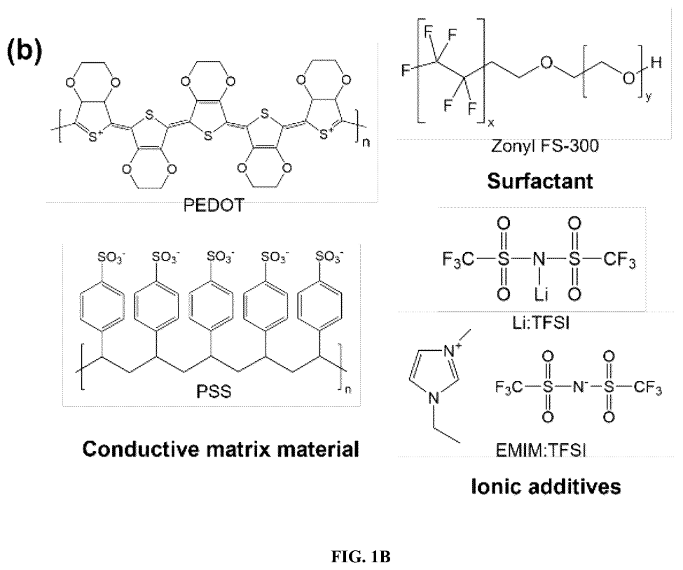

The device structure and key components of SPEARS² are illustrated in FIG. 1a and FIG. 1b. From top to bottom, a SPEARS² device consists of a stretchable substrate, a strain sensing film (SSF), an encapsulation layer with vias for electrical connection, electrode material, silver paste, silver flexible wires, and adhesive layer. The materials design of the SSF layer is important for achieving high stretchability and transparency (FIG. 1c). Poly(3,4-ethylenedioxythiophene) polystyrene sulfonate (PEDOT: PSS) was chosen as the conducting polymer due to high conductivity, optical transparency, and good processability. However, the intrinsic stretchability of PEDOT: PSS is merely ˜10%. To extend the strain sensing range, fluorinated surfactant Zonyl and ionic additives EMIM:TFSI and Li: TFSI were added. These additives serve to improve wetting of the substrate and weaken the Coulomb interaction between PEDOT and PSS and thus increase the size of conductive PEDOT domains while plasticizing the PSS matrix. This strategy extended the strain sensing range of spin-coated SSF up to 400%, by delaying crack onset and altering the crack morphology shown in FIG. 7 and Table 3. The materials design of the electrode is also important to preventing interfacial electrical failure at high strain. To ensure seamless electrical contact with the SSF, an electrode composite was developed that also contains PEDOT: PSS, but blended with a high volume fraction of carbon nanotubes for improving electrical conductivity. To accommodate the high stretchability of SSF, styrene-ethylene-butylene-styrene (SEBS) was adopted as the substrate and the encapsulation layer, which has a strain sensing range of xyz. The encapsulation layer is designed to fully encapsulate the electrode while allowing electrical contact with the SSF and Ag wires (FIG. 1a); electrode encapsulation is used to attaining high environmental stability.

The thus designed SPEARS² adheres to the target plant tissue on the two ends where the electrodes are located, leaving much of the SSF region suspended (FIG. 1a). This suspended design is intentional to avoid interference of plant tissue conductance with the resistance measurements. As the plant grows, the fixed electrodes move apart, stretching the suspended SSF to cause a resistance change. This resistance change is continuously measured with a custom-built autonomous resistance measurement system (ARMS) connected to the SPEARS² through flexible silver cables. ARMS features a data rate as high as xyz capable of detecting even nuanced changes in tissue elongation (shown later). ARMS then transmit the data wirelessly through a custom-built ZigBee system over a range of x meters.

The performance of SPEARS² is evaluated by the electromechanical response curve that plots the natural log of stretched film resistance (R) over initial resistance (R₀), or ln(R/R0), as a function of strain (FIG. 7). Stretchability is quantified by the strain sensing range (SSR), defined as the largest strain the film endures before electrical failure. Within the SSR, it was observed that ln(R/R₀) typically exhibits a linear relationship with strain:

$$ln ( \frac{R}{{R}_{0}} ) = {G}_{exp} ε \qquad ( 1 )$$

where ln (R/R0) is the sensor response, Gₑₓₚ is the sensitivity exponent, and ε is the strain of the SSF. It was shown that such exponential increase of resistance with strain is a result of crack formation, propagation and percolation (FIG. 7). Usually, a high SSR is correlated with a low Gₑₓₚ, a delayed crack onset strain (COS), and a finer and denser crack morphology.

FIG. 1a Layer-by-layer structure of the SPEARS² on a target plant surface, FIG. 1b Materials system for fabricating strain sensing films.

The monitoring of extended growth of plants benefits when the material system of the SSF layer is carefully designed for high stretchability and transparency (FIG. 1b). Poly(3,4-ethylenedioxythiophene) polystyrene sulfonate (PEDOT: PSS) is a promising candidate for this application with the advantages of easy processability, good transparency, and tunable conductivity. By adding surfactants such as Zonyl, researchers have developed highly sensitive PEDOT: PSS-based strain sensors for human motion detection with a strain sensing range (SSR) of 30%. However, this range is too small for plant growth monitoring applications, especially for fast-growing plants. To improve the stretchability of PEDOT: PSS, ionic additives (e.g., Li: TFSI, EMIM:TFSI, EMIM:DCI, EMIM:DCA, EMIM:TCB, etc.) have been blended into PEDOT: PSS matrix. These ionic additives weaken the Coulomb interaction between PEDOT and PSS in the film and increase the size of conductive PEDOT domains, and meanwhile act as a plasticizer in the PSS amorphous matrix, leading to an improvement in both conductivity and stretchability. Although ionic additive doped PEDOT: PSS films can be highly stretchable with a maximum operating strain of over 800%, they were mainly used as interconnects rather than strain sensors for quantitative elongation measurement, limited by the poor sensitivity and linearity upon strain change.

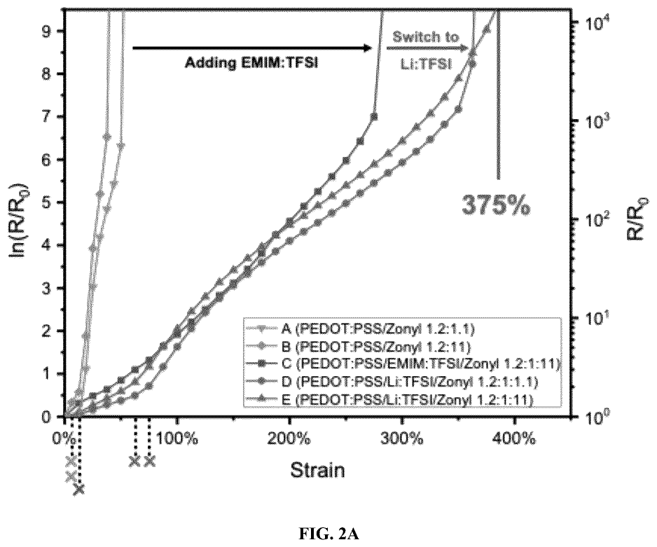

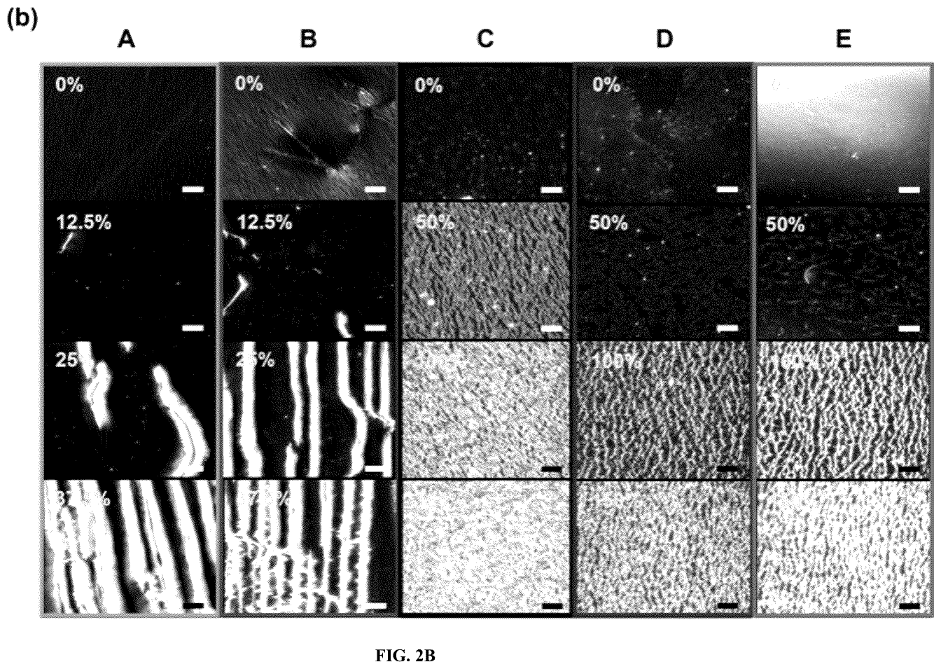

FIG. 2a Strain sensing curves (i.e., ln(R/R0)-strain curve) of five different spin-coated SSFs. The crack onset strain of each film was marked with X on the horizontal axis. FIG. 2b The crack morphology of each film when stretched to a certain strain (percentage in yellow) observed under a dark field optical microscope. Scale bar: 20 μm. The stretching and resistance measurement direction is along the short axis of the page.

Five SSFs fabricated by spin-coating on SEBS substrates from inks with different solution compositions (A, B, C, D, E, SI, Table 3) and their performances were compared. As shown in FIG. 2a.

The strain sensing curves demonstrate the significantly different electromechanical behaviors of the five SSFs. Compared with the reported value of <30% in literature, the SSR reached around 50% after adding ˜0.5 wt % Zonyl (A) in the PEDOT: PSS ink, while adding more Zonyl to ˜5 wt % (B) did not show an obvious difference. However, after blending with EMIM:TFSI, the SSR was increased to over 250% (C) and Li: TFSI doped SSFs reached a promising SSR level of around 400% (D and E). The Gₑₓₚ, on the other hand, decreased from ˜12 (A) and ˜15 (B) to 2.3 (C), 2.0 (D) and 2.2 (E), indicating a slower destruction of conductive pathways in the film upon strain after the SSFs were doped by ionic additives.

Interestingly, it was found that this drastic difference in strain sensing performance among the five SSFs can be attributed to the difference in crack development during stretching, shown by the characterizations under dark-field optical microscope (OM) (FIG. 2b). In low-SSR SSFs without ionic additives (A, B), cracks initiated as early as 12.5% strain, and the crack developed rapidly into large parallel structures destructive to conductive pathways at 37.5% strain, which leads to the high sensitivity exponent and the abrupt electrical failure at 50% strain. However, after blending with ionic additives such as EMIM:TFSI (C) and Li: TFSI (D, E), the plasticizing effect makes the films intrinsically more stretchable. Therefore, instead of abrupt, large, parallel crack morphology, these films formed a finer and less destructive winding zigzag crack morphology favorable for a slower and more uniform increase in resistance, leading to a smaller Gₑₓₚ and an elevated SSR. Furthermore, comparing the morphology difference between films C, D, and E, it was observed that at 50% strain, cracks were already substantially developed and connected in the EMIM:TFSI doped film (C), but not yet formed in Li: TFSI doped films (D, E). The slower crack initiation in the film when stretched indicates a better stretchability of the Li: TFSI-doped SSFs, leading to a further improved SSR. The crack onset strains (COS) of the 5 SSFs at which cracks are first observed under OM are marked with X's in FIG. 1a. In general, by blending ionic additives into PEDOT: PSS, films become more stretchable, suppress crack formation, and exhibit larger COSs, which improves the strain sensing performance. Because film E showed the largest SSR (400%), material composition E was selected for further SSF fabrication and investigation.

### Example 5. Printing of Highly Stretchable Strain Sensing Films

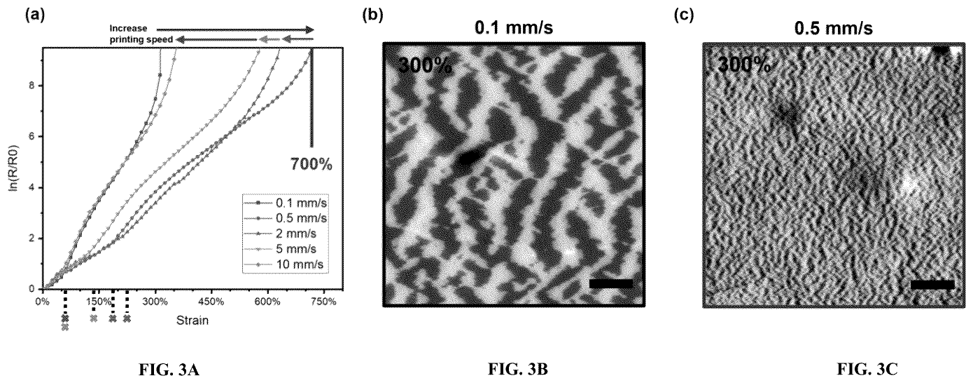

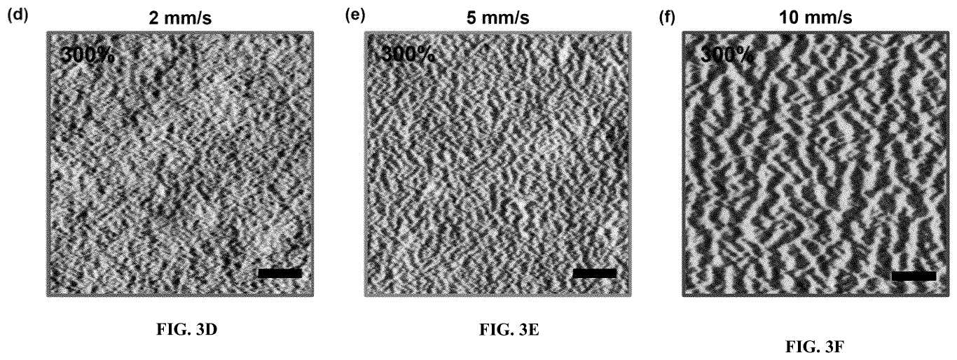

FIG. 3a Comparison of the strain sensing performance of SSFs blade coated at 5 different speeds. The crack onset strain (COS) of each film was marked with “x” on the horizontal axis. FIG. 3b-FIG. 3f Crack morphology of each film in the crack propagation stage observed under AFM (height profile. Scale bar: 5 μm). The printing, stretching, and resistance measurement is along the horizontal direction of the AFM images.

The strain sensing film comprised of PEDOT: PSS/Zonyl/Li: TFSI was processed by a meniscus-guided printing (MGP) technique, due to its ability to largely modulate film morphology and thus control the final mechanical and electronic properties of the fabricated devices. Interestingly, it was found that the SSR and Gₑₓₚ of SSF printed on SEBS substrates sensitively depend on the printing speeds and leveraged this phenomenon to significantly extend the SSR by over two-fold. As shown in FIG. 3a, at a printing speed of 0.1 mm/s, the SSR was only 300%. As the printing speed increased, the SSR reached a maximum of over 700% at 0.5 mm/s but then decreased again back to 300% at 10 mm/s. The Gₑₓₚ shows an opposite trend from that of the SSR, evident from the slope changes in the strain sensing curves as the printing speeds varied. Meanwhile, the COSs of the more stretchable SSFs (0.5 mm/s, 2 mm/s) reached above 200%, which are more than 4 times those of the less stretchable SSFs (0.1 mm/s, 10 mm/s).

To correlate the strain sensing performance with crack morphology, AFM imaging of the printed SSFs stretched to 300% was performed (FIG. 3b-f). It was found that SSFs with higher SSR and lower Gₑₓₚ (0.5 mm/s, 2 mm/s, 5 mm/s) exhibit finer crack morphologies, whereas SSFs with lower SSR and higher Gₑₓₚ (0.1 mm/s, 10 mm/s) form larger but fewer cracks. Despite the differences in crack size and area density, all films show similar “zigzag” shaped cracks featuring x degree angles formed by adjacent crack edges; this can be attributed to the orthorhombic crystal structure of PEDOT: PSS, suggesting high crystallinity in the SSFs. In previous reports, large parallel cracks and interconnected network-like cracks were often observed but neither is desirable for strain sensing applications. Because in the former case, cracks develop too fast causing abrupt electrical failure at low strain, and in the latter case, cracks develop too slowly rendering the film insensitive to strain. The zigzag-shaped cracks in the printed SSFs lie in between the two extreme cases to provide a steady and uniform reduction of conductive pathways when the films are stretched, which is favorable for balancing high SSR with good strain sensitivity. In summary, by tuning the printing speed during meniscus-guided printing of SSF, the electromechanical behaviors and crack morphologies in the films can be controlled, and a maximum SSR of over 700% has been achieved.

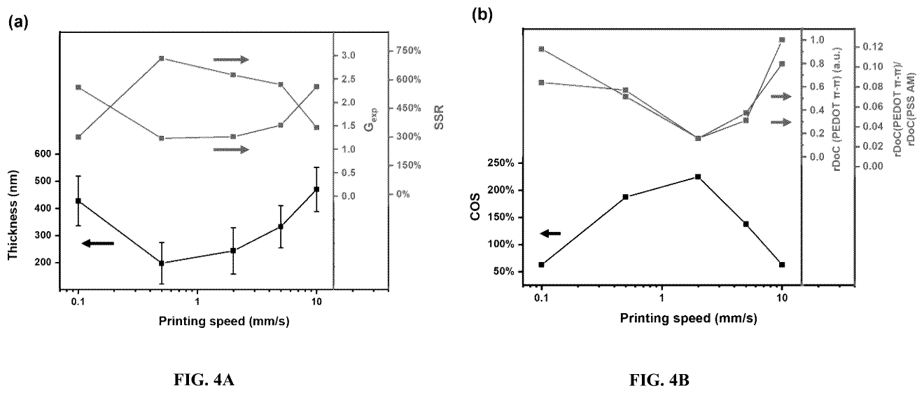

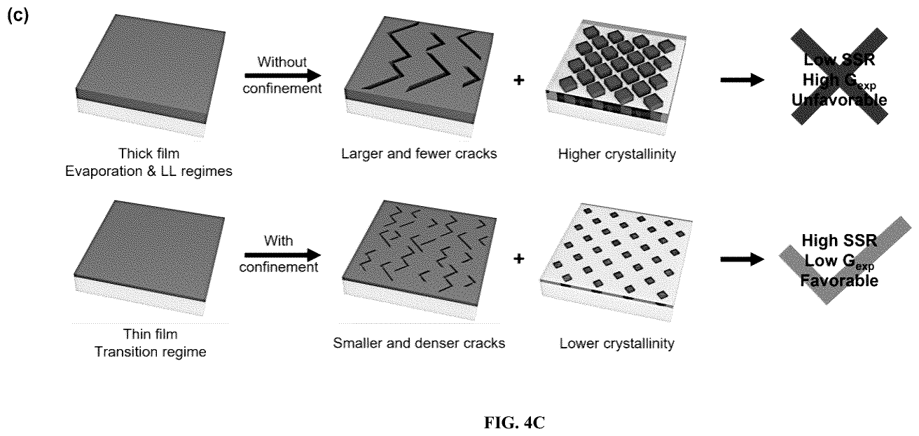

FIG. 4a Relationship between strain sensing performance (Gₑₓₚ and SSR), film thickness, and printing speed. FIG. 4b Printing speed dependence of crack onset strain (COS) and crystallinity where crystallinity is characterized by two metrics: relative degree of crystallinity (rDoc) of PEDOT π-π stacking (010) peak, and π-π/amorphous peak area ratio at chi near 90°. FIG. 4c Proposed confinement mechanism of how printing speed determines the final strain sensing performance by controlling film thickness, crack morphology, and film crystallinity.

The high SSR of SSFs printed at intermediate speeds (0.5 mm/s and 2 mm/s) results from confinement effect that suppresses PEDOT crystallization and crack development (FIG. 4). It was determined that the film thickness first decreases then increases when increasing the printing speed, and the thinnest film was obtained at 0.5 mm/s when the SSR is the largest (FIG. 4a). Such trend suggests that printing resides in the evaporation regime at speeds below 0.5 mm/s while entering the Landau-Levich (LL) regime above 0.5 mm/s, with the two regimes separated by the transition regime around 0.5 mm/s. The film thickness directly correlates with Gₑₓₚ while is inversely correlated with SSR (FIG. 4a). The thickness-dependent strain sensing performance was attributed to finer crack morphology and delayed crack initiation (higher COS) in thinner films shown in FIG. 3. This observation is consistent with prior studies that showed thinner films confine crack growth in the vertical direction, leading to nucleation of finer and denser cracks and improved SSR. Besides the difference in crack morphology, crack initiation is also much delayed in the thinnest printed films, with COSs ˜4 times that of the thickest films. However, this difference is not commonly observed in spin-coated films, which showed no clear relationship between film thickness and COS. The differences in COS of the printed films was attributed to confined crystallization in SSFs printed in the transition regime. The relative degree of crystallinity was quantified by Grazing Incidence Wide Angle X-ray Scattering (GIWAXS) shown in FIG. 7 with characteristic peaks assigned according to literature. Pole figure analysis was performed on the PEDOT π-π stacking (010) peak to extract the relative degree of crystallinity (rDoC) (see methods). A second metric was used to estimate crystallinity, by taking the ratio of the integrated, geometrically-corrected peak intensities of the PEDOT (010) π-π stacking peak vs. the amorphous ring (see methods). Both metrics clearly show that crystallinity of printed films decreases in thinner films reaching the minimum at 0.5 mm/s; this trend is inversely correlated with that of COS (FIG. 4b).

The relative degree of crystallinity (rDoC) calculated from the PEDOT π-π stacking peak following the equation below and then normalized by film thickness, which compares the degree of crystallinity of films fabricated from the same material

$$rDoC α {\int }_{0}^{\frac{π}{2}} I ( χ ) sin ( χ ) d χ \qquad ( 2 )$$

In sum, it is reasoned that for films printed in the transition regime, crack initiation is postponed, and crack development hindered in the vertical direction, owning to reduced crystallinity and thinner film thickness. This further leads to higher SSR and lower Gₑₓₚ favorable for monitoring plant growth.

### Example 6. SPEARS² Device Engineering and Testing

While an SSF with a high SSR has been successfully developed, environmental stability, sensor reliability, and reproducibility need to be addressed for robust and precise monitoring of plant growth. The environmental instability of PEDOT: PSS films in ambient conditions has been a long-standing issue because of its sensitivity to humidity, which is further magnified by the hygroscopic Li: TFSI in the SSF. While adding an encapsulation layer can make the device more stable in a humid environment, the reported encapsulation layers usually have low stretchability. Additionally, allowing electrical connection after full encapsulation while maintaining lightweight remains challenging. A recent work adopted ink-jet printing of a solvent on the encapsulation layer to make vias for electrical connection. Besides encapsulation, the electrode-SSF interface is also important to device stability and performance. Prior works have attributed interfacial instabilities to the stiffness mismatch between the electrode and the film materials. In addition to environmental stability, realizing quantitative measurement of plant growth requires good linearity in the strain sensing curve and high reproducibility of Gₑₓₚ from batch of batch.

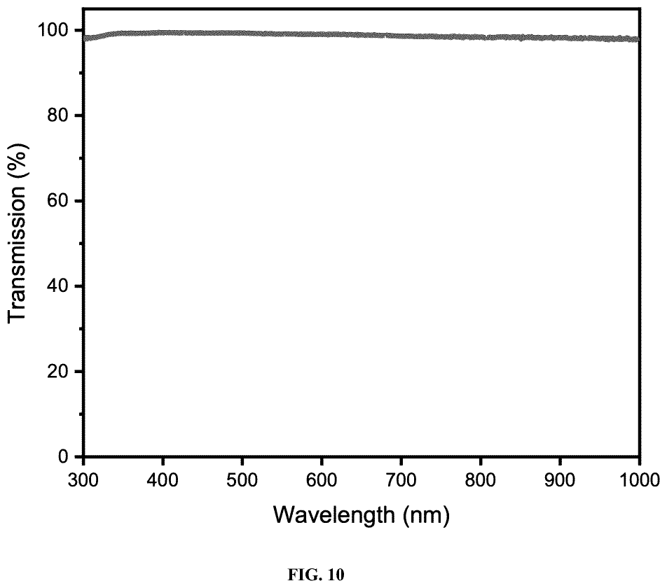

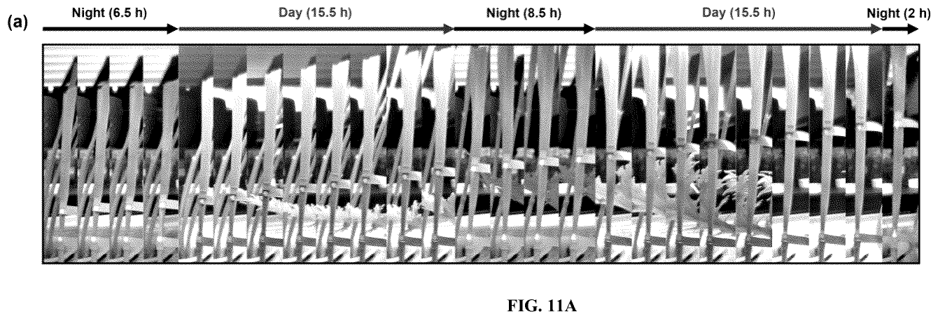

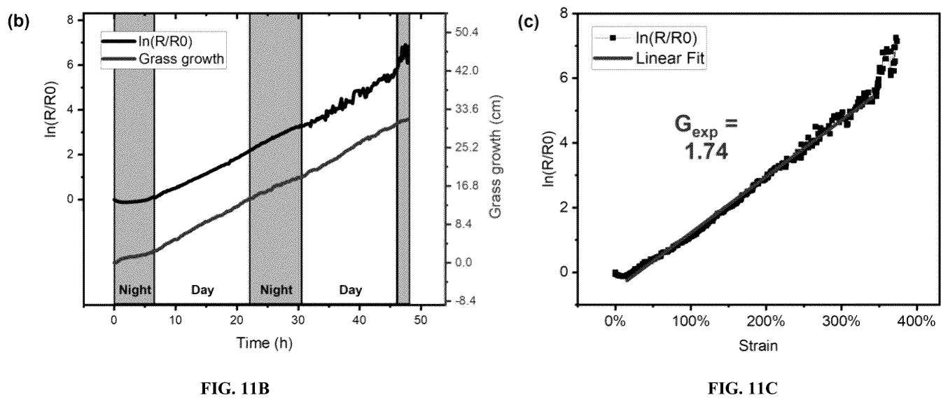

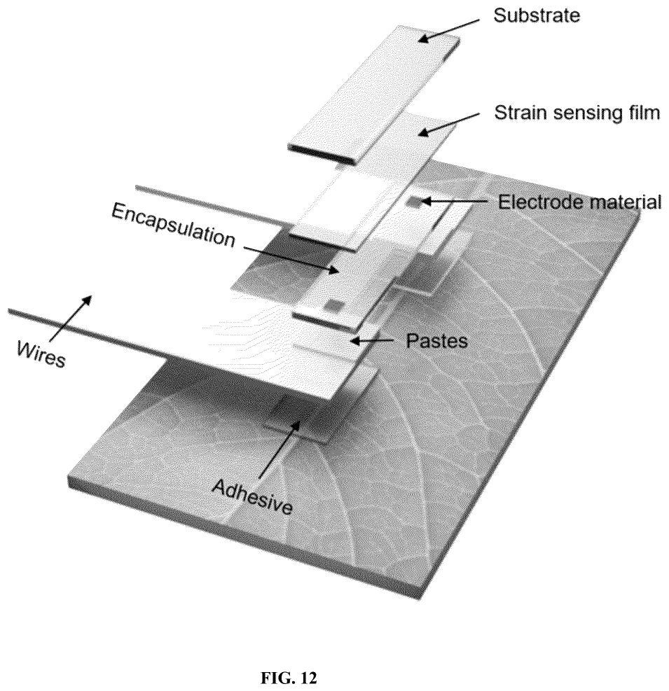

To meet the aforementioned requirements, a device fabrication process was innovated and is depicted in FIG. 5a. Steps 1-3 show SSFs printing discussed in the previous section. Steps 4-7 describe a placeholder-assisted encapsulation method for attaining environmental stability and making vias for electrical connection. A printed SSF was first cut into narrow strips of what dimension. Two PDMS placeholders (1 mm×1 mm) were then placed at the two ends of the SSF strip before a SEBS encapsulation layer was fabricated on top by MGP. The PDMS placeholders could be easily peeled off from the SSF strips after encapsulation, taking away the SEBS film on top and leaving vias for electrical connection. To facilitate electrode deposition, the encapsulated SSF strips were then separated and transferred to a support consisting of two PDMS square pads (step 8). To address the stiffness mismatch between conventional electrode materials (e.g. Ag) and the SSF, a polymer-based composite electrode ink was developed by blending PEDOT: PSS and single-walled carbon nanotubes (SWCNT) in isobutyl methyl ketone which forms a seamless interface with the SSF. The electrode ink was deposited in the vias first (step 8, black arrows), and two ultra-lightweight (each ˜20 mg) flexible silver papers were adhered to the electrode by silver paste (step 9). Finally, the device stack was peeled off from the PDMS support pads and is ready for strain sensing applications (step 10). Notably, the entire device stack is as light as ˜45 mg, and shows a transmittance as high as 98.7% in the visible light spectrum (FIG. 10), minimizing its impact on photosynthesis and plant growth. Detailed fabrication process is described in the experimental section.

To evaluate the effect of encapsulation and the PEDOT: PSS/SWCNT composite electrode on sensing performance, the fully fabricated SPEARS² was compared against two control devices where the composite electrode is replaced with silver paste, without and with encapsulation (Controls 1 & 2). Three metrics are quantitatively compared: environmental stability, linearity, and reproducibility (Table 2, FIG. 5b-FIG. 5d).

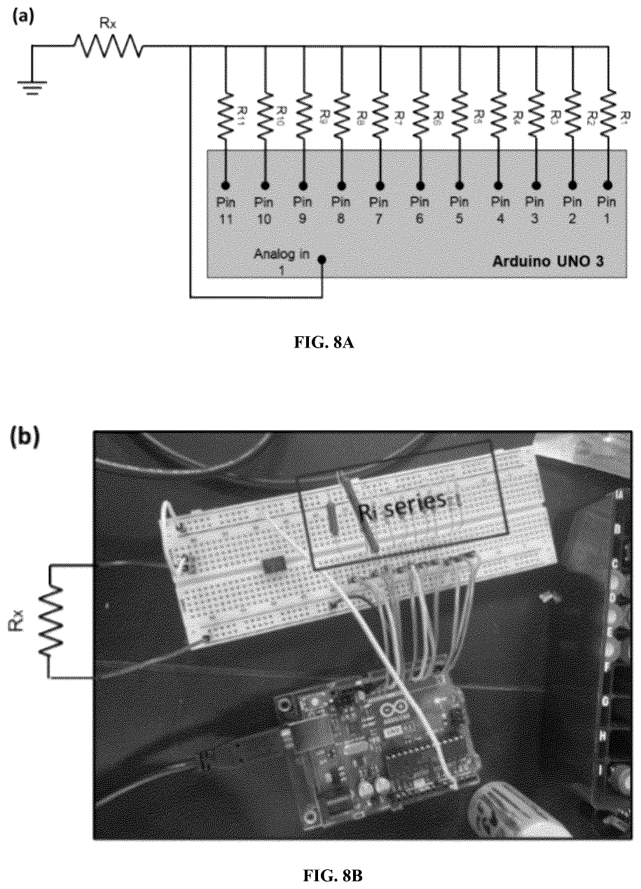

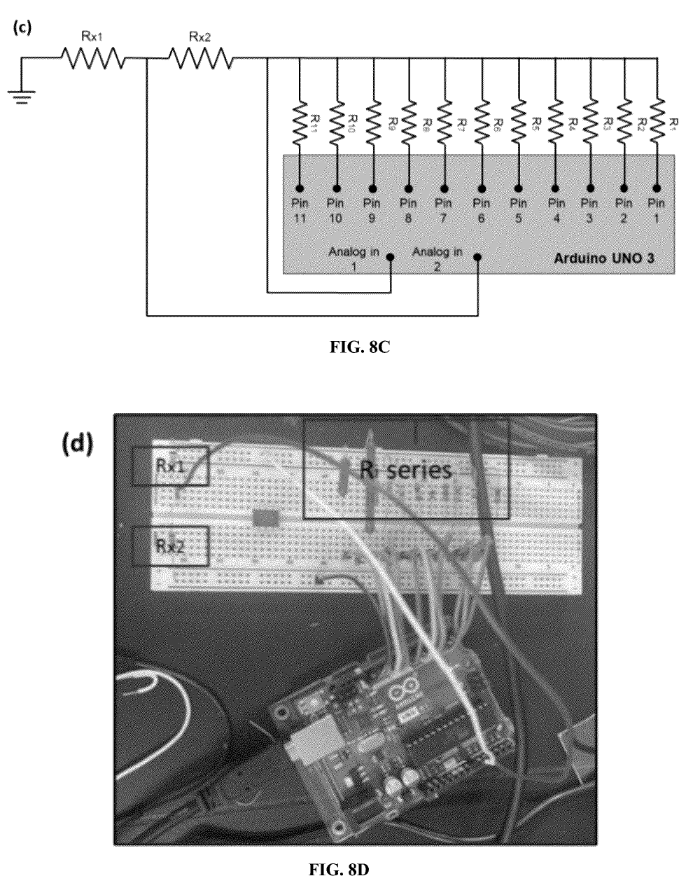

To evaluate environmental stability which shows a strong dependence on strain, a step-stretch test was developed to capture the strain-dependent environmental stability in a single test while separating the effect of strain from environmental stability on resistance change (FIG. 5b). In this test, a device was stretched by an increment of 12.5% strain and kept at that strain for 12 hours while the resistance was continuously recorded with a custom-built Autonomous Resistance Measurement System (ARMS, FIG. 8) during the entire test. The degradation rate (DR) which shows how fast the sensor response degrades under ambient conditions is defined as:

$$DR = \frac{Δ ln ( \frac{R}{{R}_{0}} )}{Δ t} \qquad ( 3 )$$

where Δt is the duration the sensor is kept at a fixed strain, and Δ ln(R/R₀) is the change in normalized resistance change at log scale over Δt. Compared with Control 1 (DR˜0.11 h⁻¹), the encapsulated device with the same electrode (Control 2) showed a decreased DR by over 2 orders of magnitudes (DR˜0.006 h-1), showing the effectiveness of the encapsulation layer in improving environmental stability. Comparing the fully encapsulated devices with the composite electrode (SPEARS²) vs. the silver paste (Control 2), composite electrode further decreased DR by ˜1 order of magnitude. This largely improved environmental stability is attributed to the protection of both the SSF and the electrodes, which significantly decreased the adsorption of water by Li: TFSI and alleviated the resistance increase caused by the swelling of the PEDOT: PSS film.

TABLE 2
SPEARS² stability, linearity and reproducibility compared to two control groups

| | | | Performance* | | | |
|---|---|---|---|---|---|---|
| | Fabrication | | Degradation | | | |
| | | Electrode | Rate | | Linearity | Reproducibility |
| Device | Encapsulation | material | (Δln(R/R0)/Δt) | | (R²) | (S/x̅) |
| Control | No | Only silver | 0.11 | h⁻¹ | N/A | 12.8% |
| 1 | | paste | | | | |
| Control | Yes | Only silver | 0.006 | h⁻¹ | 0.929 | 11.5% |
| 2 | | paste | | | | |
| SPEARS² | Yes | Black | | | | |
| | | electrode + | 0.0008 | h⁻¹ | 0.996 | 14.4% |

silver paste

*Performance parameter definitions. Degradation rate: Δln(R/R0)/Δt, measured at 12.5% strain. Linearity: R² of the ln(R/R0) − ε (strain) curve. Reproducibility: coefficient of variation of Gₑₓₚ.

FIG. 5a SPEARS² fabrication process FIG. 5b Step-stretching test of three types of SPEARS². Sensors were stretched 0.5 mm (an increase of 12.5% strain for a 4 mm-long sensor) at the end of every 12 hours while the resistance was continuously measured with ARMS FIG. 5c Comparing the strain sensing performance of silver SPEARS² and black SPEARS² at a low stretching rate (0.01 mm/min) FIG. 5d Histogram showing distribution of Gₑₓₚ of different SPEARS²

Secondly, to evaluate the sensor reliability, the linearity of sensor response curve was extracted at a low stretching rate (0.01 mm/min), which simulates how a SPEARS² works when attached to a growing plant. As shown in FIG. 5c (red curve), the strain sensing curve of Control 2 device is highly non-linear with an R2 of 0.929 undesirable for quantitative measurements. Due to the rapid resistance increase at high strain, the SSR is decreased to below 300% compared to SPEARS². The non-linearity of Control 2 device was attributed to the mismatch of mechanical properties between the hard silver electrode and the soft SSF, which may lead to interfacial electrical failure at high strain. In comparison, when adopting the composite electrode as a buffer layer between SSF and silver, the mismatch between SSF and electrode material was reduced, leading to enhanced linearity in the strain sensing curve of SPEARS² devices with R2 of 0.996 near unity and SSR of 500% (FIG. 5c, black).

Finally, the reproducibility of Gₑₓₚ was evaluated, which is important for precise and consistent measurement of plant growth, as the strain the plant experiences is calculated from ln(R/R0)/Gₑₓₚ (equation 1). Reproducibility is quantified by the coefficient of variation (CV) of Gₑₓₚ following

$$CV = \frac{S}{\overline{x}} \qquad ( 4 )$$

where S is the standard deviation and x is the mean of Gₑₓₚ which is used later to calculate strain. At least 30 samples were tested for each device type and the Gₑₓₚ distributions are shown in FIG. 5d—the Gₑₓₚ distribution of SPEARS² is right-shifted and broader (1.53±0.22) compared to that of Control 1 (1.17±0.15) and Control 2 (1.13±0.13). This is probably because the solvent (methyl isobutyl ketone) in the composite electrode ink partially dissolved the Li: TFSI in the film. Despite the broader Gₑₓₚ distribution of SPEARS², its CV (14.4%) is only slightly higher than Control 1 (12.8%) and Control 2 (11.5%), indicating good reproducibility. In sum, through fabrication process design and device engineering, a successful, highly stable, reliable, and reproducible SPEARS² was developed, which allows us to accurately track strain over a wide range.

### Example 7. Real-Time Plant Growth Monitoring with SPEARS²

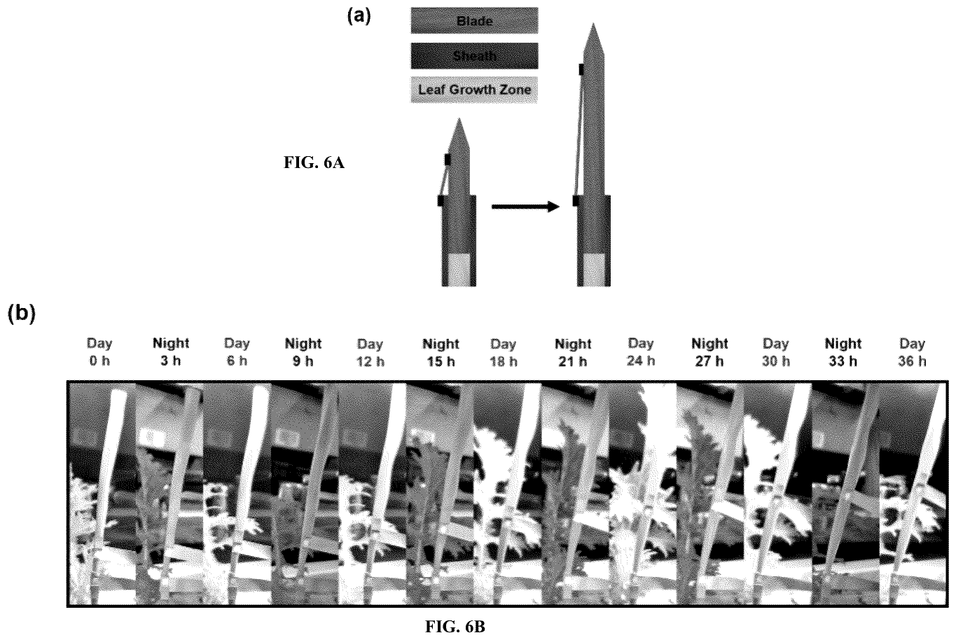

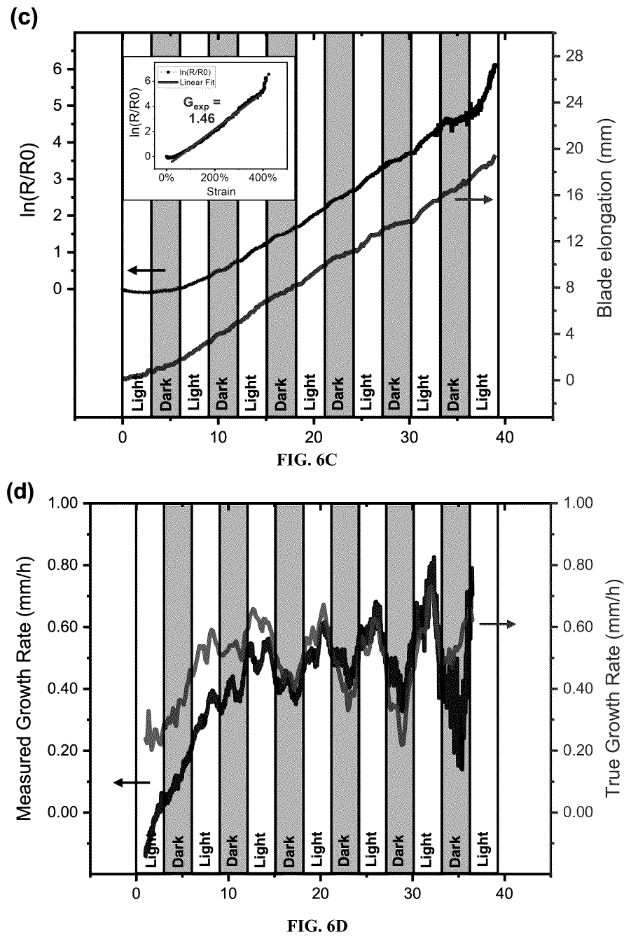

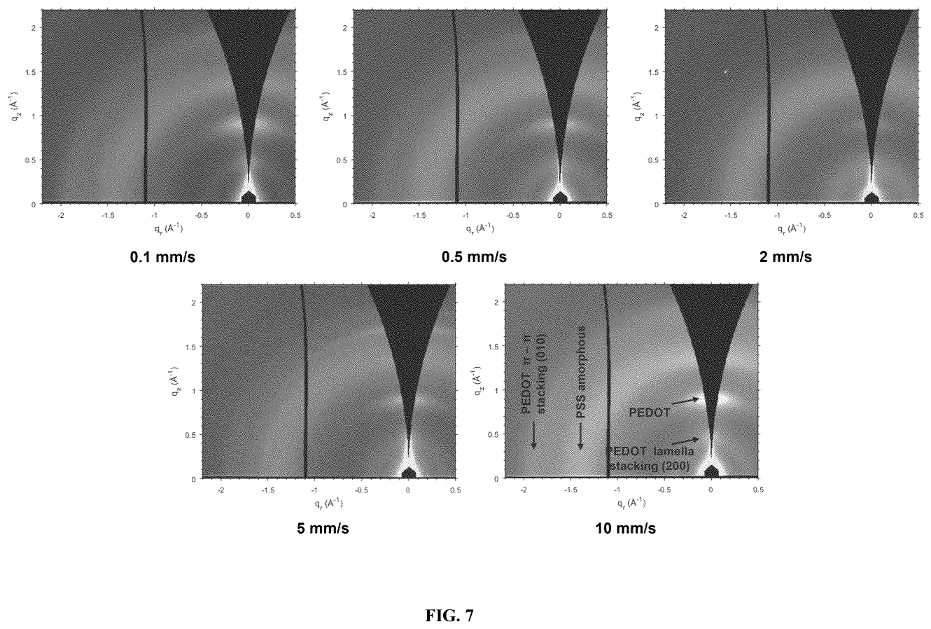

Avena Sativa, a grass species, was chosen to demonstrate the capability of SPEARS² to track the growth of small and delicate plants (FIG. 6). The mechanism of monitoring grass growth is illustrated in FIG. 8a. When a grass grows, cells are produced in the leaf growth zone enclosed by the sheath at the base of the grass, which pushes the blade upwards. SPEARS² was mounted by adhering one electrode on the sheath and the other on the blade. In this way, the middle section of SPEARS² is suspended and stretches as the blade elongates. Therefore, the resistance change only comes from stretching of the SSF due to leaf growth, not the conductance changes in the plant tissue.

FIG. 6a-FIG. 6d. Growth tracking performance of a 4.5-mm SPEARS² on grass tested with wired ARMS. FIG. 6a Schematic showing grass growth and SPEARS² stretching mechanism. FIG. 6b Camera images of mounted SPEARS² during grass growth. FIG. 6c Time-dependent sensor response compared to grass growth determined from camera images (Inset: strain sensing curve of the attached SPEARS²) FIG. 6d The measured growth rate calculated from SPEARS² response and true growth rate measured from camera images.

FIG. 6b-d shows the growth tracking performance of a SPEARS² on a grass for 39 hours with a total elongation of ˜20 mm. To observe the growth rate change in day-night cycles and capture the circadian rhythm, the lighting condition was switched on or off periodically every 3 hours. The resistance of SPEARS² was continuously measured with ARMS, and its stretching with grass growth was recorded by a camera at the same time (FIG. 6b). During the experiment, the sensor response and the blade length increased over time in parallel (FIG. 6c). It is noted that the non-ideal sensing performance during the first cycle is due to a slight pre-stretching of the sensor during transfer from the substrate to the plant, Nonetheless, over subsequent cycles, the sensor is able to capture differences of the growth rate in day vs. night manifested as the slope changes in the sensor response. Such differences is also shown in the growth curve, indicating a faithful tracking performance of SPEARS².

To evaluate how SPEARS² can quantitatively measure the grass growth rate, FIG. 6d compares the measured growth rate (MGR) calculated from the SPEARS² response (black) and the true growth rate (TGR) measured by camera imaging (red), which are defined by the following equations:

$$MGR = \frac{d [ ln ( \frac{R}{{R}_{0}} ) ]}{dt} \times \frac{{L}_{0}}{{G}_{exp}} \qquad ( 5 )$$

$$TGR = \frac{dL}{dt} \qquad ( 6 )$$

$$where \frac{d [ ln ( \frac{R}{{R}_{0}} ) ]}{dt}$$

is the time derivative of sensor response, L₀ is the initial length of the SPEARS², Gₑₓₚ is the mean sensitivity exponent obtained from the reproducibility test (i.e., 1.53), dL/dt is the time derivative of grass growth measured by camera. The strain sensing curve (inset of FIG. 6c) shows that the Gₑₓₚ of the SPEARS² tested (1.46) falls within the range of the reproducibility test (1.53±0.22). Except for the starting period, the MGR overlaps well with the TGR, demonstrating the capability of SPEARS² in precisely monitoring grass growth. Notably, peaks in MGR appear at the same locations as in the TGR curve, which indicates that SPEARS² is capable of picking up subtle changes in growth rate. SPEARS² unveils several striking features in the growth pattern. First, it took only one cycle for xyz to adapt to the artificial day-night cycles and develop a new circadian clock. This is judged from the establishment of the “pulse-like” growth pattern from Day 2. Second, it appears as if the xyz is “anticipating” the onset of day or night, given that the growth rate begins to rise (decline) even before the light is switched on (off). This is another evidence showing a new circadian rhythm has been established. Third, from Day 2 onwards, the growth rate peaks during the day and reaches lowest during the night. This pattern is strikingly similar to those observed in stomata conductance and carbon assimilation in plant, suggesting a correlation between the two. Through this example, the ability of SPEARS² to continuously and precisely track plant growth rate over extended periods was shown and could potentially lead to new fundamental understanding in plant biology.

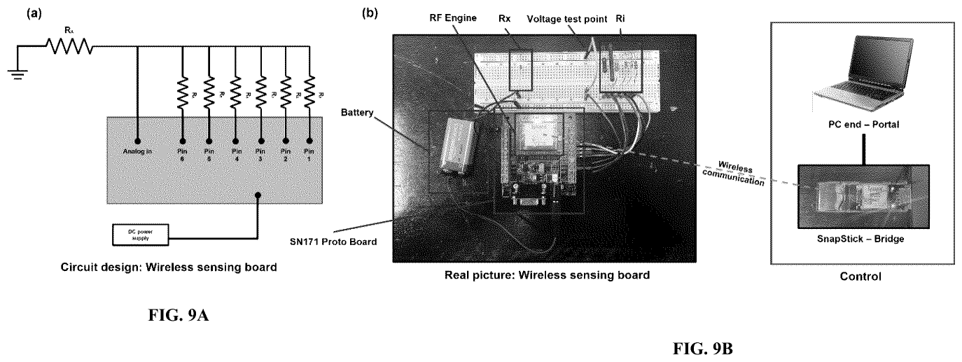

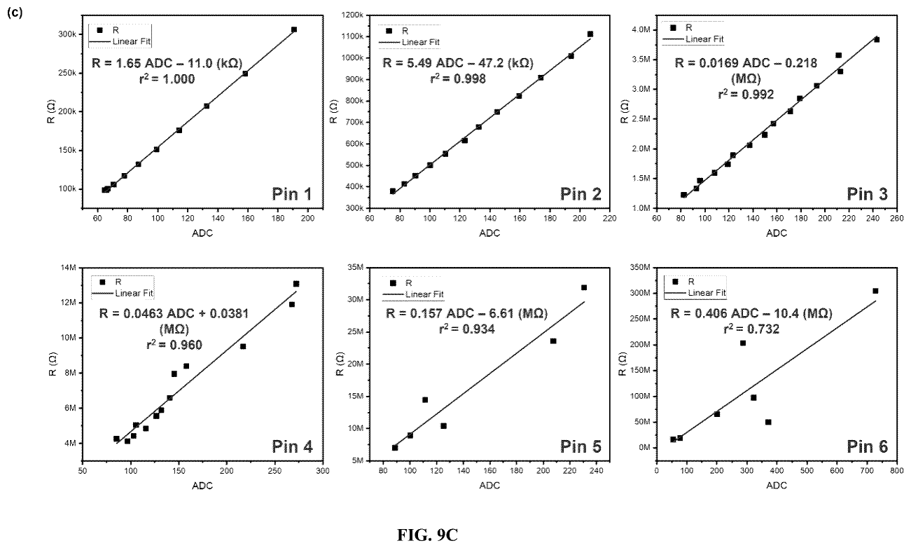

To further demonstrate remote monitoring of plant growth, a wireless ARMS (FIG. 9) was custom built and then connected with a lengthened ˜8 mm SPEARS² on the grass mm to monitor a larger growth (FIG. 11). In this experiment, the PC terminal receiving the measurement results was placed in a separate room ˜30 meters away from the experiment setup (grass, SPEARS², wireless ARMS). The sensor response of the SPEARS² was measured with the wireless ARMS in 2 days with a total grass elongation of ˜32 mm (FIG. 11a). The synchronized increase between the sensor response curve and the grass growth curve (FIG. 11b) and the good linearity of the strain sensing curve (FIG. 11c) both demonstrate the stable performance of the wireless sensing system while tracking grass growth.

### Example 8. Materials and Methods

The elastomer substrate material styrene-ethylene-butylene-styrene (SEBS) with 20% styrene content (Tuftec H1052) was obtained from Asahi Kasei. Conductive polymer poly(3,4-ethylenedioxythiophene)-poly(styrenesulfonate) (PEDOT: PSS) aqueous solution (Clevios PH1000, 1.0-1.3 wt %) was obtained from Heraeus. Zonyl FS-300 aqueous solution (40% solid) was obtained from Alpha Chemistry. The ionic additive Lithium bis(trifluoromethanesulfonyl)imide (Li: TFSI) was obtained from Sigma-Aldrich, and 1-Ethyl-3-methylimidazolium bis(trifluoromethanesulfonyl)imide (EMIM:TFSI) was obtained from SOLVIONIC SA. A certain amount of 10 mg/ml aqueous solution of the ionic additives and the as-received Zonyl FS-300 solution was added into PEDOT: PSS aqueous solution in sequence while being stirred to make different solutions for SSF fabrication.

Printable silver paste (DM-SIP-3060S) was obtained from Dycotec Materials. To fabricate ultralightweight flexible silver wires, the silver paste was spin-coated on a flexible paper substrate (the paper on the back of parafilm “M”) and annealed at 80° C. in vacuum for at least 30 minutes, followed by trimming the into ˜1.5 mm wide flexible silver paper strips, each of which weighs only ˜20 mg. PEDOT: PSS dry re-dispersible pellets, single-walled carbon nanotube (SWCNT), and 4-methyl-2-pentanone were obtained from Sigma Aldrich. 12 mg of PEDOT: PSS pellets and 12 mg of SWCNT were mixed in 1 mL of 4-methyl-2-pentanone and stirred for at least 24 hours at 80° C. to form the ink for black electrode deposition. PDMS (Dow Corning Sylgard 184, Krayden) was mixed with a 10:1 or 30:1 ratio between the base and the curing agent first, then either spin coating on glass slides (for PDMS stamps) at 800 rpm for 1 minute or molding in a petri dish (for intermediate substrate) and finally heated at 80° C. in vacuum for 2 hours.

The glass substrates for strain sensing film (SSF) fabrication were first treated with n-octyldecyltrichlorosilane (OTS, 95%, Fisher Scientific) (FIG. 5, step 1), as previously reported, to form a hydrophobic self-assembled monolayer on top of the substrate for better peeling-off of the SSF. The OTS treatment was achieved by the immersion of plasma-treated glass slides into a diluted solution of OTS (0.1 volume %) in trichloroethylene (anhydrous, >99.5%, Sigma-Aldrich) for 20 minutes at room temperature, followed by rinsing with toluene and isopropanol, drying with nitrogen, annealing at 120° C. for 20 minutes, and sonication in toluene bath for 3 minutes in sequence. For on-plant testing, seeds of cat grass (Avena Sativa) were obtained from Ferry-Morse.

### Example 9. SSF Fabrication

Firstly, the elastomer film substrate was fabricated by spin coating 200 mg/ml SEBS solution in toluene on OTS-treated glass substrates (1.25 cm×1.5 cm) at 8000 rpm for 30 seconds (FIG. 5, step 2). Next, the SEBS/glass substrates were plasma-treated for 30 minutes before usage. Then, the pre-mixed PEDOT: PSS solution with ionic additive and Zonyl was spin-coated at 1000 rpm for 1 min, or printed on the plasma-treated SEBS/glass substrates by meniscus-guided printing (MGP) (step 3), as previously reported: In a typical MGP process, an OTS-treated SiO₂ wafer was used as a blade and set at 7º and 100 μm high relative to the plasma-treated SEBS/glass substrate. After 5 μL of the ink was dispensed onto the substrate heated at 66° C., the blade was linearly translated at a certain speed, ranging from 0.1 mm/s to 10 mm/s, and sheared the ink throughout the surface of the substrate. After the spin coating MGP process, the samples were annealed at 130° C. for 15 minutes. Finally, 100 μL of deionized water or 10 mg/ml ionic additive (EMIM:TFSI or Li: TFSI) aqueous solution was dropped on the SSFs and stayed for 1 minute as a post-treatment, before the removal of the solution by spinning at 3000 rpm for 1 minute.

### Example 10. SPEARS² Fabrication

In a typical SPEARS² fabrication process, after cutting the SSF on the OTS-glass slides into several strips of ˜1.5 mm width (FIG. 5, step 4), two 1 mm×1 mm squares of 30:1 PDMS film were then put on the uniform parts of each strip as placeholders, with 5 mm space between each other (step 5). Then, to encapsulate the SSF strips, 15 μL of 50 mg/ml SEBS solution in toluene were blade coated on the SSF strips/OTS-glass substrates with the gap between the blade and the substrate set to 200 μm, while the angle remained at 7°. The substrates were kept at 90° C. during printing, and the speed of the blade was 10 mm/s (step 6). Next, the 30:1 PDMS squares were peeled off from the SSF strips, taking away the SEBS film on it and leaving two holes on the encapsulation layer for electrical connection (step 7). After cutting into the size of 8 mm×2 mm, the encapsulated SSF strips were then carefully transferred to an intermediate substrate, using a double sided-tape assisting method (step 8). Two pieces of double-sided tape were vertically stuck on a glass slide in parallel with an 8 mm gap to fix the tape position. Then, the two pieces of tape on the glass slide served as two fingers to pick up an SSF strip from the OTS-treated glass substrate. Next, the SSF sample was placed onto the intermediate substrate made from two PDMS substrates (separated with a 4-mm gap), and the two pieces of double-sided tape were cut off with a razor blade. The ink for the black electrode was deposited in the two holes on the encapsulated SSF strip and sat for 10 minutes for the solvent to evaporate (step 8). Two ultralightweight flexible silver wires were then connected to the black electrode by the same silver paste and annealed in a vacuum oven at 80° C. for at least 30 minutes (step 9). In the end, the middle part of the strain sensor that stretched during experiment was ˜4 mm. The length of the sensor can be tuned by changing the gap of the PDMS squares and the gap of the two PDMS intermediate substrates. To transfer the sensor onto a target surface, a stretchable adhesive (3M™ Medical Transfer Adhesive 4075) was first applied on the target surface, and the strain sensor can be peeled off from the PDMS substrate and attached to the subject such as grass (step 10). In some experiments, two pieces of VWR tapes were attached on the side of the sensor and the target surface to further ensure the adhesion.

### Example 11. Autonomous Resistance Measurement System (ARMS)

To realize autonomous and continuous resistance measurement and recording, a wired and wireless Automated Resistance Measurement Systems (ARMS) based on the voltage divider rule was developed. The wired ARMS was developed on an Arduino board. One (FIG. 8a, b) or two (FIG. 8c, d) strain sensor (Rx) resistance measurements ranging from 10 kΩ to 3 GΩ can be achieved at tunable data rates. For remote resistance measurement, a wireless ARMS (FIG. 9a, b) was developed by integrating the resistance measurement board with a SNAP (Subnetwork Access Protocol) wireless module, based on IEEE 802.15.4 standard. The measured ADC (Analog to Digital Converter) values are translated into resistance values through calibration curves (FIG. 9c), and can then be transmitted wirelessly to a computer, plotted in real-time via a self-developed python code for data visualization, and then saved into a CSV file. Detailed circuit and resistance measurement mechanisms can be found in SI, section 3.

### Example 12. Characterization Techniques

In situ dark field optical microscope and resistance measurement during stretching

Dark field images of the SSF at different strains were observed using an optical microscope (Eclipse Ci-POL, Nikon). An SSF was first cut into 10 mm×5 mm samples before testing. The double-sided tape assisting method described earlier was used to transfer the tailored SSF onto two glasses fixed on a motorized stage (MTS50-Z8-50 mm (1.97″) Motorized Translation Stage, Thorlabs) with an initial gap of 4 mm. Conductive wires were then bonded to the exposed part of the SSF sample on the motorized stage with silver paint (PELCOR Conductive Silver Paint, Ted Pella). Different strains were achieved by controlling the movement of the motorized linear stage at a speed of 0.1 mm/s. At each strain, a dark field image was taken, and the resistance was measured with a wide-range resistance meter (Prostat PRS-801) connected to the conductive wires. The crack onset strain (COS) was determined as the first strain where the initial cracks can be seen.

### Example 13. GIWAXS

GIWAXS measurements were done at the Argonne National Laboratory at beamline 8-ID-E, with a photon energy of 10.86 KeV, a sample-to-detector distance of 208 mm, and an incident angle of 0.14°.

### Example 14. Environmental Stability Test

To evaluate the environmental stability of the strain sensors at different strains, a step-stretch stability test was performed in ambient conditions. After the strain sensors were transferred onto the motorized stage, their resistances were measured continuously with the Arduino-based wired ARMS. The motorized stage was moved 0.5 mm (12.5% strain) each time at a speed of 0.1 mm/s after staying still for 12 hours. The measurement ended when the sensor response substantially degraded. The typical humidity in the tested environment is 30% ˜50%.

### Example 15. Response Reliability Test

The response reliability of the strain sensors at slow stretching rates mimicking the rate of plant growth was performed on a motorized stage. The gap between the moving part and the fixed part of the motorized stage was kept at 4 mm, used as the initial length while calculating strain. The strain sensors were transferred and stuck to the motorized stage with double-sided tape and connected to the Arduino-based wired ARMS. During the strain sensing performance test, the moving part of the motorized stage was moved at a speed of 0.01 mm/min (strain rate 0.25%/min) and the resistance was continuously measured during the stretching and saved after the experiments were finished.

### Example 16. Reproducibility Test

For quantitative measurement of the plant growth, the sensitivity of the strain sensor (slope in the strain sensing curve) was calculated after the strain sensing performance test of each SPEARS². The strain sensing performance test has the same procedure as the response reliability test, but at a faster stretching rate of 0.1 mm/s (strain rate 2.5%/s). The reproducibility of each kind of SPEARS² included at least the measurement of 30 samples and the Gₑₓₚ of the samples of each kind of SPEARS² were fitted and summarized.

### Example 17. Plant Cultivation and Growth Tracking Experiment

To prove the capability of the strain sensor to track plant growth, the resistance change of the strain sensor on grass (Avena Sativa) was tested. The grass was grown in a hydroponic system (Harvest Elite, Aerogarden), where the lighting conditions can be changed based on specific needs. Two pieces of 3M adhesives mentioned earlier were put on the sheath and the blade of the grass, respectively. The SPEARS² was then attached to the grass by pasting on the removable adhesive and fixing with VWR tape. To make sure no heating effect of the black electrode under light is presented in the experiment, a white pigment (Smooth-On Silc Pig Silicone Color Pigment-White) was deposited on the black electrode part of the SPEARS². The resistance of the strain sensor was continuously measured with either Arduino-based (wired) or SNAP-based (wireless) ARMS during the experiment and the grass growth was recorded with a fixed camera.

### Example 18. Composition of the Investigated Material Systems

TABLE 3
Additives and ratios of components in five different
inks for material system evaluation.

| | | | | Ionic | | |
|---|---|---|---|---|---|---|
| | | | | additive | Surfactant | |
| | Ionic | | PEDOT:PSS | weight | weight | PEDOT:PSS/Ionic |
| Ink | additive | Surfactant | weight ratio | ratio | ratio | additive/Surfactant |
| A | None | Zonyl | ~0.59% | None | ~0.54% | 1.2/None/1.1 |
| B | None | Zonyl | ~0.53% | None | ~4.89% | 1.2/None/11 |
| C | EMIM:TFSI | Zonyl | ~0.53% | ~0.44% | ~4.89% | 1.2/1/11 |
| D | Li:TFSI | Zonyl | ~0.59% | ~0.49% | ~0.54% | 1.2/1/1.1 |
| E | Li:TFSI | Zonyl | ~0.53% | ~0.44% | ~4.89% | 1.2/1/11 |

### Example 19. Arduino-Based (Wired) ARMS

To realize autonomous resistance measurement and recording, a wired automated resistance measurement system (ARMS) was developed using an Arduino UNO3 board based on the voltage divider rule to achieve continuous strain sensor resistance monitoring. FIG. 8(a) shows the ARMS circuit design for single sensor resistance measurement. To achieve wide range resistance measurement for the strain sensor (Rₓ) from 10 kΩ to 3 GΩ, eleven voltage dividing resistors (Rᵢ) named R₁ to R₁₁ (resistance value listed in the figure description) were respectively connected to 11 digital output pins (Pin 1 to Pin 11) which can power separately. When the measurement starts, each pin will be powered on in sequence to decide which Rᵢ to use to measure Rₓ. The “Analog in 1” pin measures the voltage Uₓ across Rₓ. Then, whichever resistor Rᵢ gives Uₓ closest to half of the output voltage Uₒ is selected as the voltage divider for the most accurate measurement of Rₓ. Finally, the Rₓ value can be calculated by the voltage divider rule:

$${R}_{x} = \frac{{R}_{i} \times {U}_{x}}{{U}_{o} - {U}_{x}}$$

Similarly, FIG. 8c shows the ARMS configuration that can measure two sensor resistance at the same time. The resistance of the two sensors can be calculated below:

$${R}_{x 1} = \frac{{R}_{i} \times {U}_{x 1}}{{U}_{o} - {U}_{x 2}} ,$$

$${R}_{x 2} = \frac{{R}_{i} \times ( {U}_{x 2} - {U}_{x 1} )}{{U}_{o} - {U}_{x 2}}$$

FIG. 8b and FIG. 8d shows the photos of the single and double sensor configuration of ARMS, respectively. With this design, ARMS measures and records the resistance continuously with each measurement separated by a certain time interval, which can be adjusted on demand. This automated system enables more efficient and convenient resistance measurement than manual measurement with a resistance meter, notably across a wide range from kΩ to GΩ.

### Example 20. SNAP-Based (Wireless) ARMS

For wireless resistance measurement and data transmission, a programmable wireless resistance measurement system was developed using SNAP (Subnetwork Access Protocol) based on IEEE 802.15.4 standard, which is low-power, highly reliable, and allows high data rate. The circuit design and the real picture of the wireless sensing board for resistance measurement are shown in FIG. 9a and FIG. 9b. For each selected pin (i.e., resistance measurement range), the Rx and the ADC (Analog-to-Digital Converter) value has a linear relationship in a certain region, which is summarized in FIG. 9c. The resistance-ADC value relationship was obtained by testing an SSF sample at different strains using the wireless ARMS for ADC measurement and the wired ARMS for resistance measurement. The linear fitting equations for each pin are then used as the calibration equations for the resistance measurement. During resistance measurement, the fabricated strain sensor is first connected to the wireless ARMS, which transmits the recorded data (time, ADC value, pin number) wirelessly to a PC terminal, and can be shown on the screen with a matching program Portal. Next, a customized python code reads out the ADC value and the corresponding pin, calculates the resistance value based on the calibration equations, plots the data in real-time, and finally saves the data into a CSV file. All codes used for hardware and software in the Arduino-based (wired) and SNAP-based (wireless) ARMS setup can be found at https://github.com/SiqingWang/Codes_for_ARMS.

FIG. 8a Wired ARMS circuit for single sensor measurement based on Arduino. FIG. 8b Real picture for a wired ARMS circuit for single sensor measurement. FIG. 8c Wired ARMS circuit for double sensor measurement. FIG. 8d Real picture for a wired ARMS circuit for double sensor measurement. Resistance values: R1=10 kΩ, R2=30 kΩ, R3=100 kΩ, R4=300 kΩ, R5=1 MΩ, R6=3 MΩ, R7=10 MΩ, R8=30 MΩ, R9=100 MΩ, R10=300 MΩ, R11=1 GΩ.

FIG. 9a Wireless ARMS circuit based on a SNAP wireless module, FIG. 9b Real picture of wireless ARMS circuit. Resistance values: R₁=3 MΩ, R₂=10 MΩ, R₃=30 MΩ, R₄=100 MΩ, R₅=300 MΩ, R₆=1 GΩ (c) Resistance-ADC calibration curve of each pin in the wireless ARMS.

### Example 21. Technical Achievements

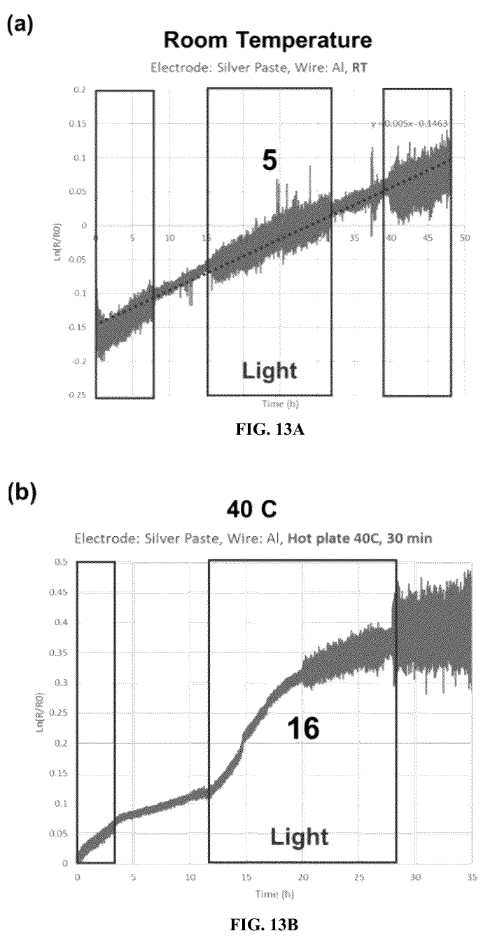

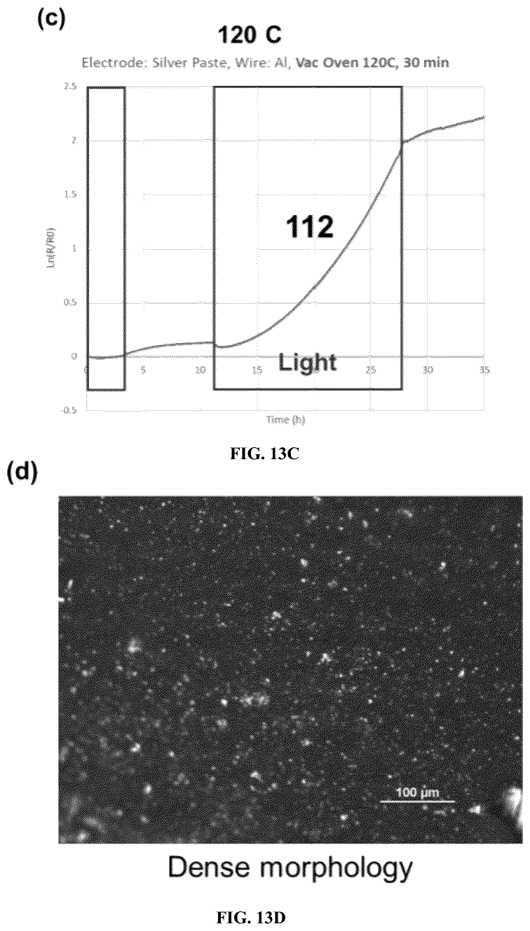

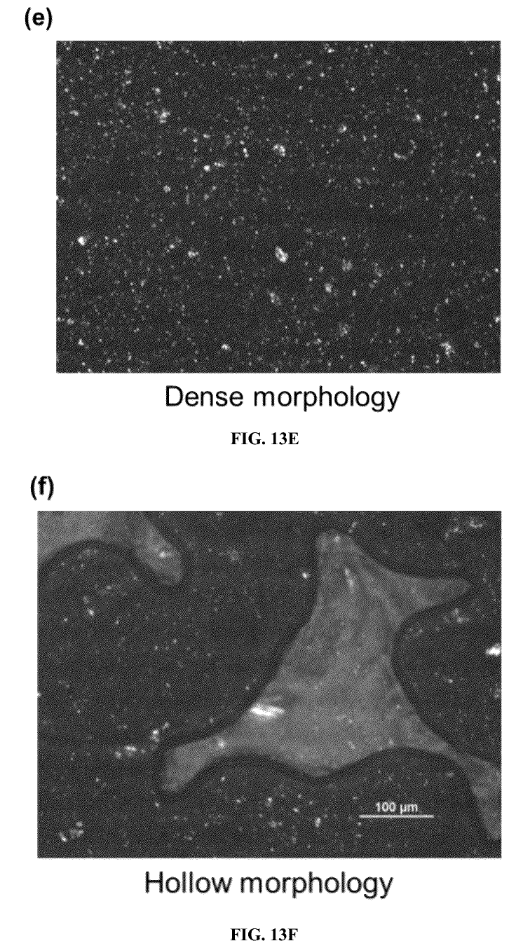

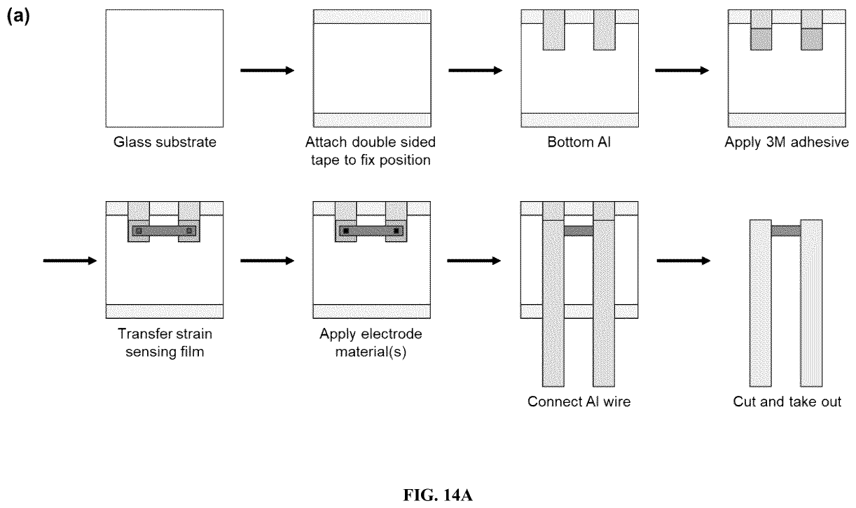

During the investigation into the effects of electrode fabrication on sensor stability under light, a significant observation was made: the curing temperature applied to the electrode after depositing the electrode material on the sensor has a crucial impact on the sensor's stability under light conditions. FIG. 13 shows how different curing temperatures affect the performance of plant strain sensors using silver paste as the electrode material under alternating light and dark conditions. As the curing temperature increased from room temperature to 120° C., there was a substantial deterioration in the stability of the strain sensor under light. This deterioration was evident from a 20-fold increase in the degradation rate, as illustrated in FIG. 13(a)-(c). This change in stability is linked to differences in the morphology of the electrode/film interface, as shown in FIG. 13(b)-(d). Higher curing temperatures lead to faster evaporation of moisture trapped within the silver paste, resulting in the formation of a hollow structure at the electrode/film interface. This structural change appears to be detrimental to the sensor's stability when exposed to light. However, it's important to note that the precise mechanism through which light affects sensor degradation still requires further investigation.

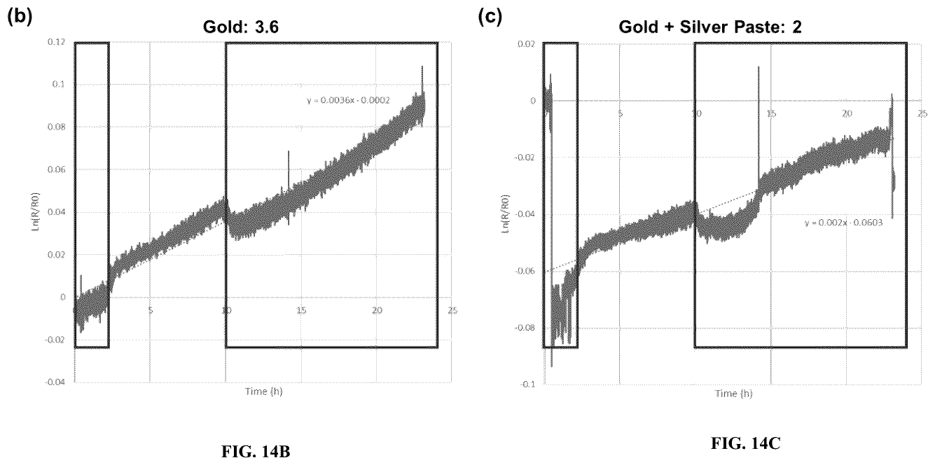

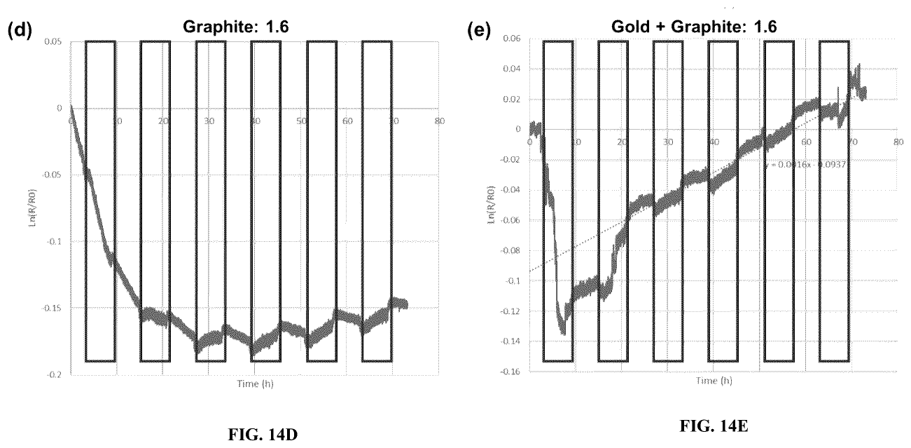

Considering the observations regarding the significance of achieving a smooth electrode/film interface and recognizing the benefits of substituting silver conductive paper with aluminum foil, along with the utilization of a lower curing temperature to enhance sensor stability under light conditions, a novel fabrication procedure was designed to implement these findings, as shown in FIG. 14a. In this modified fabrication process, the process was initiated on a glass substrate by affixing two pieces of double-sided tape. Subsequently, two pieces of aluminum foil are adhered to the upper double-sided tape, serving as a structural support for the strain sensing film. Additionally, a piece of 3M adhesive is applied to each of the aluminum foil supports. The encapsulated strain sensing film with vias for electrical connections, is then delicately transferred, making sure the connection vias are fixed onto the 3M adhesive substrate. Electrode materials are then carefully deposited into the vias, and two additional pieces of aluminum foil are affixed onto the electrode material and the lower double-sided tape. The assembly is then allowed to cure at room temperature. Upon completion of the curing process, the sensor can be readily separated from the substrate with minimal damage to the electrode segment. FIG. 14b-FIG. 14d provide examples of electrode materials utilized in the assembly of a wrapped-Al strain sensor. These materials include gold, gold/silver paste combination, graphite, and gold/graphite combination. The observations from the figures reveal that the employment of gold and graphite as the electrode material results in a consistent degradation rate, irrespective of light or dark conditions. Furthermore, this combination exhibits a notably low degradation rate, measuring only 1.6 ×10⁻³ h⁻¹.

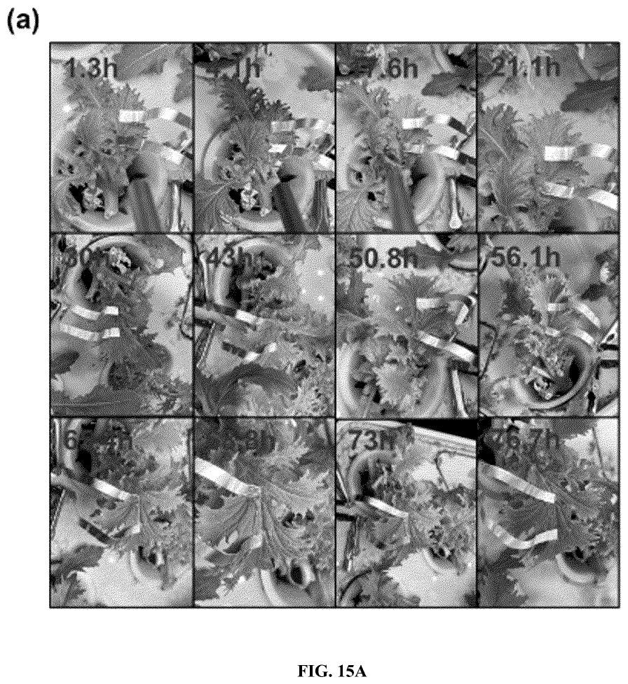

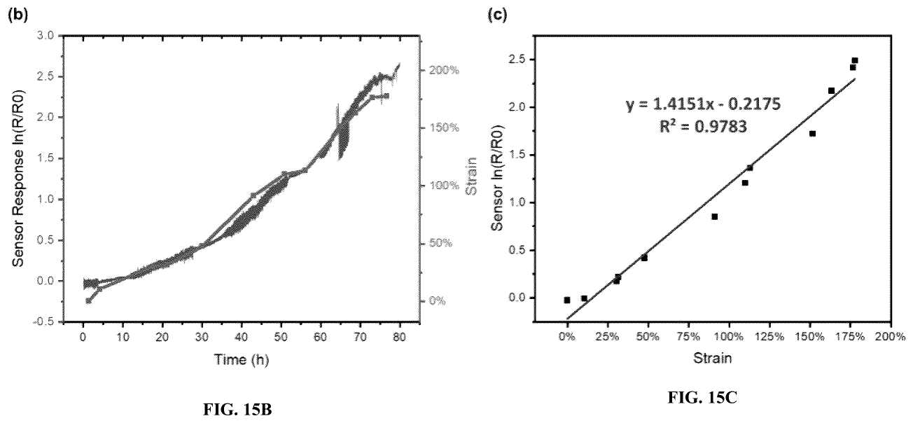

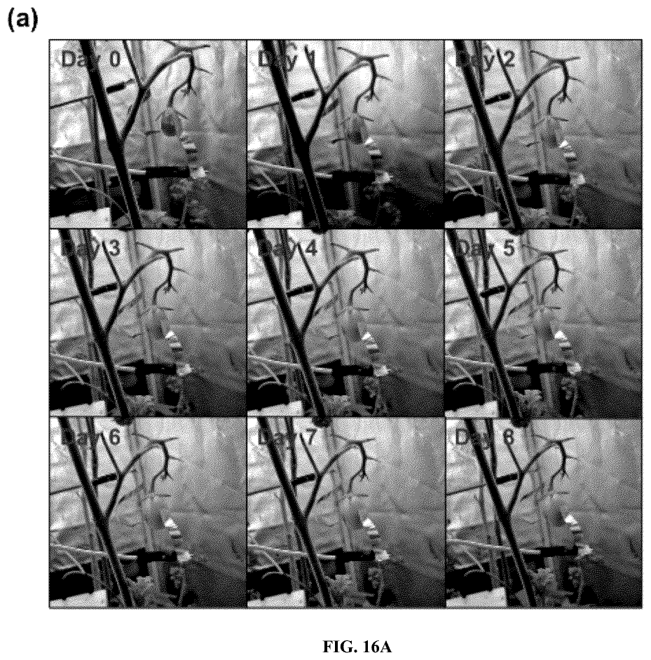

Subsequently, the newly developed strain sensor was applied to monitor the growth of a Mizuna leaf, as depicted in FIG. 15a-FIG. 15e. During leaf growth, images were captured with the camera oriented vertically toward the leaf surface (FIG. 15a), and the sensor response was continuously recorded throughout the experiment. FIG. 15b presents a graphical representation of the change of the sensor response (in black) alongside the calculated strain of the sensor (in red) derived from the camera images as a function of time. Notably, these two curves exhibit a high degree of congruence, indicating that the strain sensor functions normally on Mizuna, irrespective of the presence of light. The strain sensing curve, as depicted in FIG. 15c, displays a notable linear trend characterized by a slope of 1.41 and a R-square value of 0.98. The strain sensor functions well even after 75 hours of continuous stretching in conjunction with a Mizuna leaf.

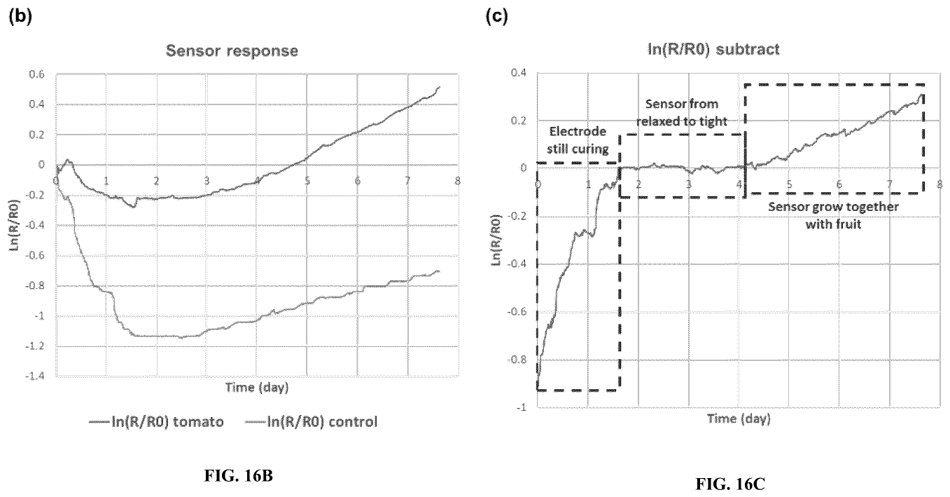

Finally, the light-stable wrapped-Al strain sensor was applied to monitor a young tomato fruit cultivated within a plant growth tent, as illustrated in FIG. 16a-FIG. 16c. The experiment consisted of two groups: an experimental group, where the strain sensor was partially wrapped around a tomato fruit, and a control group, in which a strain sensor was placed stationary on the ground. FIG. 16a presents a sequence of camera images capturing the strain sensor growing together with the tomato fruit over an 8-day monitoring period. FIG. 16b depicts the sensor responses for both the experimental (tomato) group and the control group. The control group's sensor response also exhibited an increase over time, attributed to the gradual degradation of the strain sensor. However, it is evident that the increase in sensor response for the experimental group is more pronounced, demonstrating the influence of fruit growth on altering the strain sensor's response. Upon further analysis, three distinctive stages were observed when subtracting the sensor response of the experimental group from that of the control group and resetting the zero point, as shown in FIG. 16c. In the initial 1.5 days, the observed inconsistency between the two sensors is attributable to the ongoing curing process of the electrode material, leading to disparities in their responses. From day 1.5 to day 4, the sensor response, after subtraction, remains relatively stable. This phase may result from the strain sensor's incomplete conformation to the fruit surface. As the fruit initially grows, the strain sensor attempts to adapt but does not induce any additional strain, resulting in an unaltered sensor response. Commencing from day 4, a notable increase is observed as the strain sensor begins to grow together with the developing fruit. The sensor response, after subtraction, exhibits an increase, indicating the strain of the strain sensor induced by the growing fruit.

All publications mentioned herein are incorporated by reference to the extent they support the present invention.

### REFERENCES

A number of patents and publications are cited above in order to more fully describe and disclose the invention and the state of the art to which the invention pertains. Full citations for these references are provided below. Each of these references is incorporated herein by reference in its entirety into the present disclosure, to the same extent as if each individual reference was specifically and individually indicated to be incorporated by reference.
ADDIN EN.REFLIST (1) Bar-On, Y. M.; Phillips, R.; Milo, R. The biomass distribution on Earth. Proceedings of the National Academy of Sciences 2018, 115 (25), 6506-6511. DOI: 10.1073/pnas.1711842115 (acccessed 2023 Mar. 16).
(2) van Dijk, M.; Morley, T.; Rau, M. L.; Saghai, Y. A meta-analysis of projected global food demand and population at risk of hunger for the period 2010-2050. Nature Food 2021, 2 (7), 494-501. DOI: 10.1038/s43016-021-00322-9.
(3) Ehrlich, J.; Massa, G.; Wheeler, R.; Gill, T.; Quincy, C.; Roberson, L.; Binsted, K.; Morrow, R. Plant Growth Optimization by Vegetable Production System in HI-SEAS Analog Habitat; 2017. DOI: 10.2514/6.2017-5143.
(4) James, J.; Maheshwar P, M. Plant growth monitoring system, with dynamic user-interface. In IEEE Region 10 Humanitarian Technology Conference, 2016.
(5) Singh, R.; Srivastava, S.; Mishra, R. AI and IoT Based Monitoring System for Increasing the Yield in Crop Production. In International Conference on Electrical and Electronics Engineering, 2020.
(6) Wu, L.; Long, Y.; Sun, H.; Liu, N.; Wang, W.; Dong, Q. Length Measurement of Potato Leaf using Depth Camera. IFAC-PapersOnLine 2018, 51 (17), 314-320. DOI: 10.1016/j.ifacol.2018.08.197.
(7) Nugroho, A. P.; Fadilah, M. A. N.; Wiratmoko, A.; Azis, Y. A.; Efendi, A. W.; Sutiarso, L.; Okayasu, T. Implementation of crop growth monitoring system based on depth perception using stereo camera in plant factory. In IOP Conf. Series: Earth and Envirorunental Science, 2020.
(8) Apelt, F.; Breuer, D.; Nikoloski, Z.; Stitt, M.; Kragler, F. Phytotyping (4D): a light-field imaging system for non-invasive and accurate monitoring of spatio-temporal plant growth. Plant J 2015, 82 (4), 693-706. DOI: 10.1111/tpj.12833.
(9) Wang, S.; Xiao, P.; Liang, Y.; Zhang, J.; Huang, Y.; Wu, S.; Kuo, S.-W.; Chen, T. Network cracks-based wearable strain sensors for subtle and large strain detection of human motions. Journal of Materials Chemistry C 2018, 6 (19), 5140-5147. DOI: 10.1039/c8tc00433a.
(10) Souri, H.; Bhattacharyya, D. Highly sensitive, stretchable and wearable strain sensors using fragmented conductive cotton fabric. Journal of Materials Chemistry C 2018, 6 (39), 10524-10531. DOI: 10.1039/c8tc03702g.
(11) Chun, S.; Choi, Y.; Park, W. All-graphene strain sensor on soft substrate. Carbon 2017, 116, 753-759. DOI: 10.1016/j.carbon.2017.02.058.
(12) Fan, X.; Wang, N.; Yan, F.; Wang, J.; Song, W.; Ge, Z. A Transfer-Printed,

Stretchable, and Reliable Strain Sensor Using PEDOT: PSS/Ag NW Hybrid Films Embedded into Elastomers. Advanced Materials Technologies 2018, 3 (6). DOI: 10.1002/admt.201800030.
(13) Pan, F.; Chen, S.-M.; Li, Y.; Tao, Z.; Ye, J.; Ni, K.; Yu, H.; Xiang, B.; Ren, Y.; Qin, F.; et al. 3D Graphene Films Enable Simultaneously High Sensitivity and Large Stretchability for Strain Sensors. Advanced Functional Materials 2018, 28 (40). DOI: 10.1002/adfm.201803221.
(14) Dauzon, E.; Lin, Y.; Faber, H.; Yengel, E.; Sallenave, X.; Plesse, C.; Goubard, F.; Amassian, A.; Anthopoulos, T. D. Stretchable and Transparent Conductive PEDOT: PSS-Based Electrodes for Organic Photovoltaics and Strain Sensors Applications. Advanced Functional Materials 2020, 30 (28). DOI: 10.1002/adfm.202001251.
(15) Yu, Y.; Zhai, Y.; Yun, Z.; Zhai, W.; Wang, X.; Zheng, G.; Yan, C.; Dai, K.; Liu, C.; Shen, C. Ultra-Stretchable Porous Fiber-Shaped Strain Sensor with Exponential Response in Full Sensing Range and Excellent Anti-Interference Ability toward Buckling, Torsion, Temperature, and Humidity. Advanced Electronic Materials 2019, 5 (10). DOI: 10.1002/aelm.201900538.
(16) Sun, Z.; Yang, S.; Zhao, P.; Zhang, J.; Yang, Y.; Ye, X.; Zhao, X.; Cui, N.; Tong, Y.; Liu, Y.; et al. Skin-like Ultrasensitive Strain Sensor for Full-Range Detection of Human Health Monitoring. ACS Applied Materials & Interfaces 2020, 12 (11), 13287-13295. DOI: 10.1021/acsami.9b21751.
(17) Xia, H.; Xia, Y.; Ye, Y.; Qian, L.; Shi, G. Simultaneous Wireless Strain Sensing and Energy Harvesting From Multiple Piezo-Patches for Structural Health Monitoring Applications. IEEE Transactions on Industrial Electronics 2019, 66 (10), 8235-8243. DOI: 10.1109/tie.2018.2883277.
(18) Occhiuzzi, C.; Paggi, C.; Marrocco, G. Passive RFID Strain-Sensor Based on Meander-Line Antennas. IEEE Transactions on Antennas and Propagation 2011, 59 (12), 4836-4840. DOI: 10.1109/tap.2011.2165517.
(19) Tetsu, Y.; Yamagishi, K.; Kato, A.; Matsumoto, Y.; Tsukune, M.; Kobayashi, Y.; Fujie, M. G.; Takeoka, S.; Fujie, T. Ultrathin epidermal strain sensor based on an elastomer nanosheet with an inkjet-printed conductive polymer. Applied Physics Express 2017, 10 (8). DOI: 10.7567/apex.10.087201.
(20) Roh, E.; Hwang, B.-U.; Kim, D.; Kim, B.-Y.; Lee, N.-E. Stretchable, Transparent, Ultrasensitive, and Patchable Strain Sensor for Human-Machine Interfaces Comprising a Nanohybrid of Carbon Nanotubes and Conductive Elastomers. ACS Nano 2015, 9 (6), 6252-6261. DOI: 10.1021/acsnano.5b01613.
(21) Zhou, J.; Guo, X.; Xu, Z.; Wu, Q.; Chen, J.; Wu, J.; Dai, Y.; Qu, L.; Huang, Z. Highly sensitive and stretchable strain sensors based on serpentine-shaped composite films for flexible electronic skin applications. Composites Science and Technology 2020, 197. DOI: 10.1016/j.compscitech.2020.108215.
(22) Lee, H.; Seong, B.; Moon, H.; Byun, D. Directly printed stretchable strain sensor based on ring and diamond shaped silver nanowire electrodes. RSC Advances 2015, 5 (36), 28379-28384. DOI: 10.1039/c5ra01519g.
(23) Tang, W.; Yan, T.; Ping, J.; Wu, J.; Ying, Y. Rapid Fabrication of Flexible and Stretchable Strain Sensor by Chitosan-Based Water Ink for Plants Growth Monitoring. Advanced Materials Technologies 2017, 2 (7). DOI: 10.1002/admt.201700021.
(24) Nassar, J. M.; Khan, S. M.; Villalva, D. R.; Nour, M. M.; Almuslem, A. S.; Hussain, M. M. Compliant plant wearables for localized microclimate and plant growth monitoring. npj Flexible Electronics 2018, 2 (1). DOI: 10.1038/s41528-018-0039-8.
(25) Tang, W.; Yan, T.; Wang, F.; Yang, J.; Wu, J.; Wang, J.; Yue, T.; Li, Z. Rapid fabrication of wearable carbon nanotube/graphite strain sensor for real-time monitoring of plant growth. Carbon 2019, 147, 295-302. DOI: 10.1016/j.carbon.2019.03.002.
(26) Zhao, Y.; Gao, S.; Zhu, J.; Li, J.; Xu, H.; Xu, K.; Cheng, H.; Huang, X. Multifunctional Stretchable Sensors for Continuous Monitoring of Long-Term Leaf Physiology and Microclimate. ACS Omega 2019, 4 (5), 9522-9530. DOI: 10.1021/acsomega.9b01035.
(27) Zhang, C.; Zhang, C.; Wu, X.; Ping, J.; Ying, Y. An integrated and robust plant pulse monitoring system based on biomimetic wearable sensor. npj Flexible Electronics 2022, 6 (1), 43. DOI: 10.1038/s41528-022-00177-5.
(28) Taroni, P. J.; Santagiuliana, G.; Wan, K.; Calado, P.; Qiu, M.; Zhang, H.; Pugno, N. M.; Palma, M.; Stingelin-Stutzman, N.; Heeney, M.; et al. Toward Stretchable Self-Powered Sensors Based on the Thermoelectric Response of PEDOT: PSS/Polyurethane Blends. Advanced Functional Materials 2018, 28 (15), 1704285, https://doi.org/10.1002/adfm.201704285. DOI: https://doi.org/10.1002/adfm.201704285 (acccessed 2022 Oct. 19).
(29) Han, L.; Yan, L.; Wang, M.; Wang, K.; Fang, L.; Zhou, J.; Fang, J.; Ren, F.; Lu, X. Transparent, Adhesive, and Conductive Hydrogel for Soft Bioelectronics Based on Light-Transmitting Polydopamine-Doped Polypyrrole Nanofibrils. Chemistry of Materials 2018, 30 (16), 5561-5572. DOI: 10.1021/acs.chemmater.8b01446.
(30) Lei, Z.; Wu, P. A highly transparent and ultra-stretchable conductor with stable conductivity during large deformation. Nature Communications 2019, 10 (1), 3429. DOI: 10.1038/s41467-019-11364-w.
(31) Wang, Y.; Zhu, C.; Pfattner, R.; Yan, H.; Jin, L.; Chen, S.; Molina-Lopez, F.; Lissel, F.; Liu, J.; Rabiah, N. I.; et al. A highly stretchable, transparent, and conductive polymer. Sci Adv 2017, 3 (3), e1602076. DOI: 10.1126/sciadv.1602076.
(32) Wu, Q.; Wei, J.; Xu, B.; Liu, X.; Wang, H.; Wang, W.; Wang, Q.; Liu, W. A robust, highly stretchable supramolecular polymer conductive hydrogel with self-healability and thermo-processability. Scientific Reports 2017, 7 (1), 41566. DOI: 10.1038/srep41566.
(33) Kayser, L. V.; Lipomi, D. J. Stretchable Conductive Polymers and Composites Based on PEDOT and PEDOT: PSS. Adv Mater 2019, 31 (10), e1806133. DOI: 10.1002/adma.201806133.
(34) Savagatrup, S.; Chan, E.; Renteria-Garcia, S. M.; Printz, A. D.; Zaretski, A. V.; O'Connor, T. F.; Rodriquez, D.; Valle, E.; Lipomi, D. J. Plasticization of PEDOT: PSS by Common Additives for Mechanically Robust Organic Solar Cells and Wearable Sensors. Advanced Functional Materials 2015, 25 (3), 427-436. DOI: 10.1002/adfm.201401758.
(35) Fan, X.; Wang, N.; Wang, J.; Xu, B.; Yan, F. Highly sensitive, durable and stretchable plastic strain sensors using sandwich structures of PEDOT: PSS and an elastomer. Materials Chemistry Frontiers 2018, 2 (2), 355-361, 10.1039/C7QM00497D. DOI: 10.1039/C7QM00497D.
(36) Kee, S.; Kim, H.; Paleti, S. H. K.; El Labban, A.; Neophytou, M.; Emwas, A.-H.; Alshareef, H. N.; Baran, D. Highly Stretchable and Air-Stable PEDOT: PSS/Ionic Liquid Composites for Efficient Organic Thermoelectrics. Chemistry of Materials 2019, 31 (9), 3519-3526. DOI: 10.1021/acs.chemmater.9b00819.
(37) Teo, M. Y.; Kim, N.; Kee, S.; Kim, B. S.; Kim, G.; Hong, S.; Jung, S.; Lee, K. Highly Stretchable and Highly Conductive PEDOT: PSS/Ionic Liquid Composite Transparent Electrodes for Solution-Processed Stretchable Electronics. ACS Appl Mater Interfaces 2017, 9 (1), 819-826. DOI: 10.1021/acsami.6b11988.
(38) Park, K. S.; Kwok, J. J.; Dilmurat, R.; Qu, G.; Kafle, P.; Luo, X.; Jung, S. H.; Olivier, Y.; Lee, J. K.; Mei, J.; et al. Tuning conformation, assembly, and charge transport properties of conjugated polymers by printing flow. Sci Adv 2019, 5 (8), eaaw7757. DOI: 10.1126/sciadv.aaw7757.
(39) Lee, G.-H.; Lee, Y. R.; Kim, H.; Kwon, D. A.; Kim, H.; Yang, C.; Choi, S. Q.; Park, S.; Jeong, J.-W.; Park, S. Rapid meniscus-guided printing of stable semi-solid-state liquid metal microgranular-particle for soft electronics. Nature Communications 2022, 13 (1), 2643. DOI: 10.1038/s41467-022-30427-z.
(40) Aasmundtveit, K. E.; Samuelsen, E. J.; Pettersson, L. A. A.; Inganäs, O.; Johansson, T.; Feidenhans'l, R. Structure of thin films of poly(3,4-ethylenedioxythiophene). Synthetic Metals 1999, 101 (1), 561-564. DOI: https://doi.org/10.1016/S0379-6779 (98) 00315-4.
(41) Hamasha, M. M.; Alzoubi, K.; Switzer, J. C.; Lu, S.; Desu, S. B.; Poliks, M. A study on crack propagation and electrical resistance change of sputtered aluminum thin film on poly ethylene terephthalate substrate under stretching. Thin Solid Films 2011, 519 (22), 7918-7924. DOI: 10.1016/j.tsf.2011.06.062.
(42) Le Berre, M.; Chen, Y.; Baigl, D. From Convective Assembly to Landau-Levich Deposition of Multilayered Phospholipid Films of Controlled Thickness. Langmuir 2009, 25 (5), 2554-2557. DOI: 10.1021/la803646e.
(43) Balar, N.; O'Connor, B. T. Correlating Crack Onset Strain and Cohesive Fracture Energy in Polymer Semiconductor Films. Macromolecules 2017, 50 (21), 8611-8618. DOI: 10.1021/acs.macromol.7b01282.
(44) Müller, A. J.; Arnal, M. L.; Trujillo, M.; Lorenzo, A. T. Super-nucleation in nanocomposites and confinement effects on the crystallizable components within block copolymers, miktoarm star copolymers and nanocomposites. European Polymer Journal 2011, 47 (4), 614-629. DOI: https://doi.org/10.1016/j.eurpolymj.2010.09.027.
(45) Papananou, H.; Perivolari, E.; Chrissopoulou, K.; Anastasiadis, S. H. Tuning polymer crystallinity via the appropriate selection of inorganic nanoadditives. Polymer 2018, 157, 111-121. DOI: https://doi.org/10.1016/j.polymer.2018.10.018.
(46) Mahmood, A.; Wang, J.-L. A Review of Grazing Incidence Small- and Wide-Angle X-Ray Scattering Techniques for Exploring the Film Morphology of Organic Solar Cells. Solar RRL 2020, 4 (10), 2000337, https://doi.org/10.1002/solr.202000337. DOI: https://doi.org/10.1002/solr.202000337 (acccessed 2023 Mar. 21).
(47) Biessmann, L.; Kreuzer, L. P.; Widmann, T.; Hohn, N.; Moulin, J. F.; Muller-Buschbaum, P. Monitoring the Swelling Behavior of PEDOT: PSS Electrodes under High Humidity Conditions. ACS Appl Mater Interfaces 2018, 10 (11), 9865-9872. DOI: 10.1021/acsami.8b00446.
(48) Wang, Y. F.; Sekine, T.; Takeda, Y.; Yokosawa, K.; Matsui, H.; Kumaki, D.; Shiba, T.; Nishikawa, T.; Tokito, S. Fully Printed PEDOT: PSS-based Temperature Sensor with High Humidity Stability for Wireless Healthcare Monitoring. Sci Rep 2020, 10 (1), 2467. DOI: 10.1038/s41598-020-59432-2.
(49) Liu, N.; Fang, G.; Wan, J.; Zhou, H.; Long, H.; Zhao, X. Electrospun PEDOT: PSS-PVA nanofiber based ultrahigh-strain sensors with controllable electrical conductivity. Journal of Materials Chemistry 2011, 21 (47), 18962-18966, 10.1039/C1JM14491J. DOI: 10.1039/C1JM14491J.
(50) Zhao, P.; Zhang, R.; Tong, Y.; Zhao, X.; Zhang, T.; Tang, Q.; Liu, Y. Strain-Discriminable Pressure/Proximity Sensing of Transparent Stretchable Electronic Skin Based on PEDOT: PSS/SWCNT Electrodes. ACS Applied Materials & Interfaces 2020, 12 (49), 55083-55093. DOI: 10.1021/acsami.0c16546.
(51) Molina-Lopez, F.; Gao, T. Z.; Kraft, U.; Zhu, C.; Öhlund, T.; Pfattner, R.; Feig, V. R.; Kim, Y.; Wang, S.; Yun, Y.; et al. Inkjet-printed stretchable and low voltage synaptic transistor array. Nature Communications 2019, 10 (1), 2676. DOI: 10.1038/s41467-019-10569-3.
(52) Kim, D. W.; Kong, M.; Jeong, U. Interface Design for Stretchable Electronic Devices. Adv Sci (Weinh) 2021, 8 (8), 2004170. DOI: 10.1002/advs.202004170 From NLM PubMed-not-MEDLINE.
(53) Kavanova, M.; Grimoldi, A. A.; Lattanzi, F. A.; Schnyder, H. Phosphorus nutrition and mycorrhiza effects on grass leaf growth. P status- and size-mediated effects on growth zone kinematics. Plant Cell Environ 2006, 29 (4), 511-520. DOI: 10.1111/j.1365-3040.2005.01428.x.
(54) Creux, N.; Harmer, S. Circadian Rhythms in Plants. Cold Spring Harb Perspect Biol 2019, 11 (9). DOI: 10.1101/cshperspect.a034611 From NLM Medline.
(55) Hennessey, T. L.; Field, C. B. Circadian Rhythms in Photosynthesis 1: Oscillations in Carbon Assimilation and Stomatal Conductance under Constant Conditions. Plant Physiology 1991, 96 (3), 831-836. DOI: 10.1104/pp. 96.3.831 (acccessed Dec. 22, 2022).
(56) Hennessey, T. L.; Freeden, A. L.; Field, C. B. Environmental effects on circadian rhythms in photosynthesis and stomatal opening. Planta 1993, 189 (3), 369-376. DOI: 10.1007/BF00194433.
(57) Wheeler, R. M.; Fitzpatrick, A. H.; Tibbitts, T. W. Potatoes as a Crop for Space Life Support: Effect of CO2, Irradiance, and Photoperiod on Leaf Photosynthesis and Stomatal Conductance. Frontiers in Plant Science 2019, 10, Original Research.
(58) Wheeler, R. M.; Mackowiak, C. L.; Yorio, N. C.; Sager, J. C. Effects of CO2on Stomatal Conductance: Do Stomata Open at Very High CO2Concentrations? Annals of Botany 1999, 83 (3), 243-251. DOI: https://doi.org/10.1006/anbo.1998.0813.

We claim:

1. A strain sensor, said sensor comprising:
a substrate layer,
a strain sensing film (SSF) layer, wherein said SSF layer comprises a conductive polymer, a surfactant, and an ionic additive,
an encapsulation layer,
at least one electrode material,
one or more pastes (e.g., a silver (Ag) paste),
one or more wires (e.g., a silver (Ag) flexible wire),
one or more adhesive layers,
wherein said SSF layer has a stretchability of about 1 to about 1000% and a transparency of 0% to about 99%.

2. The strain sensor of claim 1, wherein said SSF layer has a stretchability of about 1-1000% and a transparency of 70% to 99%.

3. The strain sensor of claim 1, wherein said SSF layer has a stretchability of about 200-1000% and a transparency of 0% to 99%.

4. The strain sensor of claim 1, wherein said SSF layer has a stretchability of about 200-1000% and a transparency of about 70% to about 99%.

5. A strain sensor, said sensor comprising:
a styrene-ethylene-butylene-styrene (SEBS) substrate layer
a strain sensing film (SSF) layer, wherein said SSF comprises a conductive polymer, a surfactant, and an ionic additive,
an encapsulation layer comprising SEBS,
at least one electrode material comprising a composite of poly(3,4-ethylenedioxythiophene) polystyrene sulfonate (PEDOT: PSS) and single walled carbon nanotube (SWCNT);
one or more pastes,
one or more wires,
one or more adhesive layers,
wherein said conductive polymer comprises a poly(3,4-ethylenedioxythiophene) polystyrene sulfonate (PEDOT: PSS),
wherein said surfactant comprises Zonyl,
wherein said ionic additive comprises Li: TFSI, EMIM:TFSI, EMIM:DCI, EMIM:DCA, and/or EMIM:TCB, and
wherein said SSF layer has a stretchability of about 1-1000% and a transparency of 0% to 99%.

6. The sensor of claim 1, wherein said substrate layer comprises one or more components chosen from Polydimethylsiloxane (PDMS), Polyethylene (PE), Polyethylene Terephthalate (PET), Polypropylene (PP), Polystyrene (PS), Natural Rubber, Styrene-ethylene-butylene-styrene (SEBS), Ecoflex, Polyether Block Amide (PEBA), Thermoplastic Polyurethane (TPU), and Thermoplastic Vulcanizate (TPV).

7. The sensor of claim 1, wherein said conductive polymer is chosen from poly(3,4-ethylenedioxythiophene) polystyrene sulfonate (PEDOT: PSS), poly(3,4-ethylenedioxythiophene) (PEDOT) derivatives and copolymers, poly(3,4-propylenedioxythiophene) (PProDOT) derivatives and copolymers, poly(3,4-alkylenedioxythiophene)s (e.g., poly(3,4-dialkylthiophene)s, poly(3,4-cycloalkylthiophene)s, poly(3,4-dialkoxythiophene)s, poly(3,4-alkylenedioxythiophene) s) derivatives and copolymers, polyaniline (PANI), polythiophene (PTh), Polypyrrole (PPy)

8. The sensor of claim 1, wherein said ionic additive is chosen from inorganic salts (e.g., NaClO₄, LiClO₄), organic salts (e.g., Bis(trifluoromethane) sulfonimide lithium salt, 4-(3-Butyl-1-imidazolio)-1-butanesulfonic acid triflate, 1-Butyl-3-methylimidazolium octyl sulfate, Zinc di[bis(trifluoromethyl sulfonyl)imide], 4-(3-Butyl-1-imidazolio)-1-butanesulfonate, 1-Ethyl-3-methylimidazolium-bis(trifluoromethylsulfonyl)imide, Methyl-trioctylammonium bis(trifluoromethylsulfonyl imide, Trihexyltetradecyl phosphonium bis(2-(4-trimethylpentyl)phosphinate, 1-Butyl-3-methylpyridinium bis(trifluormethylsulfonyl)imide, Dioctyl sulfosuccinatesodium salt, Sodium dodecylbenzenesulfonate, Dodecylbenzenesulfonic acid, 1-Ethyl-3-methylimidazolium 4,5-dicyanoimidazolate, 1-Ethyl-3-methylimidazolium dicyanamide, and 1-Ethyl-3-methylimidazolium tetracyanoborate).

9. The sensor of claim 1, wherein said surfactant is chosen from ionic surfactants (e.g., Sodium lauryl sulfate (SLS), Sodium laureth sulfate (SLES), Ammonium lauryl sulfate (ALS), Ammonium laureth sulfate (ALES), Sodium stearate, Sodium Dodecyl Sulfate (SDS), Potassium cocoate), and non-ionic surfactants (e.g., Zonyl, Triton X, Tween, polysorbates, sorbitans, PEG).

10. The sensor of claim 1, wherein said encapsulation layer comprises one or more components chosen from Polydimethylsiloxane (PDMS), Polyethylene (PE), Polyethylene Terephthalate (PET), Polypropylene (PP), Polystyrene (PS), Natural Rubber, Styrene-ethylene-butylene-styrene (SEBS), Ecoflex, Polyether Block Amide (PEBA), Thermoplastic Polyurethane (TPU), and Thermoplastic Vulcanizate (TPV).

11. The sensor of claim 1, wherein said electrode material is chosen from poly(3,4-ethylenedioxythiophene) polystyrene sulfonate (PEDOT: PSS), poly(3,4-ethylenedioxythiophene) (PEDOT) derivatives and copolymers, poly(3,4-propylenedioxythiophene) (PProDOT) derivatives and copolymers, carbon based materials (e.g., carbon nanotubes, carbon black, graphite, graphene), metals (e.g., silver, copper, gold), and the mixture thereof.

12. The sensor of claim 1, wherein said paste is a conductive paste (e.g. a paste comprising a conductive material such as silver, carbon, copper, or gold).

13. The sensor of claim 1, wherein said wire is a conductive wire (e.g. a wire comprising a metal such as silver, carbon, copper, gold, aluminum, or platinum; metal conductive paper, which may be made by spin coating metal paste on a paper, for instance, silver conductive paper).

14. The sensor of claim 1, wherein said adhesive layer (e.g., double sided flexible tapes, medical adhesives, or other tapes; adhesive layer may be removable).

15. A method for monitoring a plant elongation, said method comprising: attaching a strain sensor of claim 1 on a plant tissue, and measuring a resistance of said strain sensor.

16. The method of claim 15, wherein said monitor occurs remotely.

17. A method of fabricating a strain sensor for monitoring a plant elongation, said method comprising:
(a) spin coating a layer of a SEBS substrate on a slide,
(b) blade coating a layer of SSF onto said SEBS layer, wherein said SSF layer is optionally subjected to an annealing process or a solution treatment process,
(c) optionally blade coating a second SEBS layer on said SSF layer,
(d) attaching an electrode material to said second SEBS layer,
(e) optionally applying said electrode on a paste and wherein said paste is attached to said second SEBS layer,
(f) peeling an assembled film off from said slide with a water-soluble tape (WST), wherein said assembled film comprises said SEBS substrate layer, said SSF layer, and said electrode,
(g) cutting said assembled film into strips,
(h) optionally pasting said strips on an adhesive layer, wherein said adhesive layers is an adhesive layer with a water-soluble tape (WST), and optionally removing said adhesive layers, and
(i) connecting said electrode with a conductive wire for resistance measurement. wherein said electrode comprises PEDOT: PSS, Li: TFSI, single-walled carbon nanotubes (SWCNT), or any combination thereof.
